SEQUENCE LISTING (14 sequences)

SEQ ID NO: 1 - DNA, 687 bp, Homo sapiens
atgctgtcggtgatggtctcttcctctctggtcctgatcgtcttttttctaggcgcttccgaggaggcgaagccggcgacgacgacgataaagaatacaaagccgcagtgtcgtccagaggattacgcgaccagattgcaagatctccgcgtcacctttcatcgagtaaaacctacgttggtaggtcacgtaggtacggtttattgcgacggtctttcttttccgcgtgtcgggtgacgtagttttcctcttgtagcaacgtgaggacgactactccgtgtggctcgacggtacggtggtcaaaggctgttggggatgcagcgtcatggactggttgttgaggcggtatctggagatcgtgtttcccgcaggcgaccacgtctatcccggactcaagacggaattgcatagtatgcgctcgacgctagaatccatctacaaagacatgcggcaatgtgtaagtgtctctgtggcggcgctgtccgcacagaggtaacaacgtgttcatagcacgctgttttacttttgtcgggctcccagcctctgttaggttgcggagataagtccgtgattagtcggctgtctcaggaggcggaaaggaaatcggataacggcacgcggaaaggtctcagcgagttggacacgttgtttagccgtctcgaagagtatctgcactcgagaaagtag

SEQ ID NO: 2 - DNA, 503 bp, Homo sapiens
tgcggcgatgctgtcggtgatggtctcttcctctctggtcctgatcgtcttttttctaggcgcttccgaggaggcgaagccggcgacgacgacgataaagaatacaaagccgcagtgtcgtccagaggattacgcgaccagattgcaagatctccgcgtcacctttcatcgagtaaaacctacgttggtaggtcacgtaggtacggtttattgcgacggtctttcttttccgcgtgtcgggtgacgtagttttcctcttgtagcaacgtgaggacgactactccgtgtggctcgacggtacggtggtcaaaggctgttggggatgcagcgtcatggactggttgttgaggcggtatctggagatcgtgtttcccgcaggcgaccacgtctatcccggactcaagacggaattgcatagtatgcgctcgacgctagaatccatctacaaagacatgcggcaatgtgtaagtgtctctgtggcggcgctgtccgcacagaggtaa

SEQ ID NO: 3 - protein, 176 aa, Homo sapiens
MLSVMVSSSLVLIVFFLGASEEAKPATTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 4 - protein, 140 aa, Homo sapiens [sequence omitted]

SEQ ID NO: 5 - DNA, 18 bp, Homo sapiens
gcaccatcctcctcttcc

SEQ ID NO: 6 - DNA, 19 bp, Homo sapiens
ggcctctgataaccaagcc

SEQ ID NO: 7 - DNA, 37 bp, Homo sapiens
ggtcactagtgacgcttgtatgatgaccatgtaccga

SEQ ID NO: 8 - DNA, 25 bp, Homo sapiens
gatagtcgcgggtacaggggactct

SEQ ID NO: 9 - DNA, 21 bp, Homo sapiens
aagtgagttctgtcgggtgct

SEQ ID NO: 10 - DNA, 22 bp, Homo sapiens
gtgacaccagagaatcagagga

SEQ ID NO: 11 - DNA, 22 bp, Homo sapiens
gtgagtccgaggagatgaaatg

SEQ ID NO: 12 - DNA, 22 bp, Homo sapiens
ctcgtagataggcagcatgaac

SEQ ID NO: 13 - DNA, 18 bp, Homo sapiens
aagagaggcatcctcacc

SEQ ID NO: 14 - DNA, 18 bp, Homo sapiens
tacatggctggggtgttg

(19) United States
(12) Patent Application Publication
(10) Pub. No.: US 2018/0128830 A1
(43) Pub. Date: May 10, 2018

(54) DETECTION OF HUMAN CYTOMEGALOVIRUS IN BREAST CANCER

(71) Applicants: Juliet V. Spencer, San Mateo, CA (US); Vivian Poi Young, San Francisco, CA (US)

(72) Inventors: Juliet V. Spencer, San Mateo, CA (US); Vivian Poi Young, San Francisco, CA (US)

(21) Appl. No.: 15/566,999

(22) PCT Filed: Apr. 15, 2016
(86) PCT No.: PCT/US16/27947

§ 371 Date: Oct. 16, 2017

Related U.S. Application Data
(60) Provisional application No. 62/148,625, filed on Apr. 16, 2015.

(51) Int. Cl. G01N 33/569; C12Q 1/6886; C12Q 1/686; G01N 33/574; C12Q 1/70; A61P 35/04

(52) U.S. Cl. CPC G01N 33/56983; C12Q 1/6886; C12Q 1/686; G01N 33/57488; C12Q 1/70; G01N 33/57415; C12Q 2600/118; C12Q 2600/158; C07K 2317/00; G01N 2800/56; G01N 2800/54; A61P 35/04

(57) ABSTRACT

Provided herein, inter alia, are methods for predicting the occurrence of metastasis in an individual affected with breast cancer as well as methods for determining the risk of metastasis in individuals diagnosed with breast tumors by assaying for the presence of viral interleukin-10 (cmvIL-10) in individuals diagnosed with or suspected of having breast cancer that are seronegative for human cytomegalovirus (HCMV).

### CROSS-REFERENCE TO RELATED APPLICATIONS

This application claims priority to U.S. Provisional Patent Application No. 62/148,625, filed Apr. 16, 2015, the disclosure of which is incorporated by reference herein in its entirety.

### FIELD OF INVENTION

This invention relates generally to the field of methods for screening individuals with breast cancer for the presence of human cytomegalovirus (HCMV) and assessing the risk of metastasis based on the existence of the same. The invention also relates to forming ex vivo complexes of HCMV and probe(s) that detect HCMV.

### BACKGROUND

Breast cancer is the second leading cause of cancer deaths in the United States (Key et al., (2001) Lancet Oncol 2: 133-140). Many cancer patients do not die from local complications of their primary tumor growth, but rather from the malignant spread of the tumor. Approximately 30% of patients diagnosed with a solid tumor already have a clinically detectable metastasis, and for the remaining 70%, metastases are continually being formed throughout the life of the tumor (Tuszynski (2001) Pathol Oncol Res 7: 14-23). While there are recognized genetic, environmental, and behavioral risk factors associated with breast cancer, little is known about the connection between infectious agents and breast cancer development or progression.

As such, there is a particular need for improved methods for identifying and assessing the risk for metastatic potential in individuals diagnosed with, or thought to have, breast cancer or breast tumors. Methods such as these would be an invaluable asset to health care practitioners for monitoring and choosing the most appropriate course of treatment in these individuals.

Throughout this specification, various patents, patent applications and other types of publications (e.g., journal articles, electronic database entries, etc.) are referenced. The disclosure of all patents, patent applications, and other publications cited herein are hereby incorporated by reference in their entirety for all purposes.

### SUMMARY

The invention provided herein discloses, inter alia, methods for predicting or determining the occurrence of metastasis in an individual affected with breast cancer as well as methods for determining the risk of metastasis in individuals diagnosed with or thought to have breast tumors based on the expression of viral interleukin-10 (cmvIL-10) in individuals that are seronegative for human cytomegalovirus (HCMV).

Accordingly, in some aspects, provided herein are methods for predicting the occurrence of metastasis in an individual affected with breast cancer, the method comprising: detecting the presence of viral interleukin 10 (cmvIL-10) in a biological sample provided by the individual, wherein the presence of cmvIL-10 indicates that the breast cancer has metastasized and wherein the individual is seronegative for HCMV. In other aspects, provided herein are methods for determining the risk of metastasis in an individual affected with a breast tumor, the method comprising: detecting the presence of viral interleukin 10 (cmvIL-10) in a biological sample provided by the individual, wherein the presence of cmvIL-10 indicates that the individual is at increased risk for breast cancer metastasis and wherein the individual is seronegative for HCMV. In some embodiments of any of the embodiments disclosed herein, the sample is selected from the group consisting of a blood sample, a tissue sample, a urine sample, a saliva sample, a semen sample, a tear sample, or a breast milk sample. In some embodiments of any of the embodiments disclosed herein, cmvIL-10 is detected by detecting a cmvIL-10 nucleic acid in the sample. In some embodiments, the cmvIL-10 nucleic acid is DNA. In some embodiments, the cmvIL-10 DNA is detected by PCR or Southern Blotting. In some embodiments, the cmvIL-10 nucleic acid is RNA. In some embodiments, the cmvIL-10 RNA is detected by RT-PCR, Northern Blotting, in situ hybridization, microarray, or RNase protection assay. In some embodiments of any of the embodiments disclosed herein, the cmvIL-10 is detected by detecting a cmvIL-10 protein in the sample. In some embodiments, the vIL-10 protein is detected by Western Blotting, immunoprecipitation, immunocytochemistry, immunohistochemistry, immunoelectron microscopy, radioimmunoassay, Enzyme-Linked ImmunoSpot (ELISPOT) assay, 2D gel electrophoresis, or enzyme-linked immunosorbent assay (ELISA). In some embodiments, the cmvIL-10 protein is detected by ELISA. In some embodiments, the antibody used in the ELISA is a polyclonal antibody. In some embodiments, the antibody used in the ELISA assay is a monoclonal antibody. In some embodiments of any of the embodiments disclosed herein, said individual is pre- or post-menopausal. In some embodiments of any of the embodiments disclosed herein, said individual has been diagnosed as having breast cancer and said method is used to determine if said breast cancer has recurred or advanced. In some embodiments of any of the embodiments disclosed herein, said individual has not been previously diagnosed as having breast cancer. In some embodiments of any of the embodiments disclosed herein, cmvIL-10 is LAcmvIL-10. In some embodiments, the method further comprises detecting upregulation of at least one gene selected from the group consisting of plasminogen activator inhibitor 1 (PAI-1), urokinase plasminogen activator (uPA), urokinase plasminogen activator receptor (uPAR), and matrix metalloproteinase-3 (MMP-3). In some embodiments of any of the embodiments disclosed herein, the method further comprises detecting downregulation of the gene missing-in-metastasis (MTSS). In some embodiments of any of the embodiments disclosed herein, the method further comprises detecting CXCR4-mediated calcium signaling. In some embodiments of any of the embodiments disclosed herein, the method further comprises detecting chemotaxis toward CXCL12.

In further aspects, provided herein is a kit for detecting human cytomegalovirus (HCMV) in a sample provided by an individual diagnosed with breast cancer comprising: a) a probe for detecting the presence of viral interleukin 10 (cmvIL-10) in the sample; and b) one or more buffers and/or reagents, wherein the individual is seronegative for HCMV. In some embodiments, the probe is selected from the group consisting of a nucleic acid probe or an antibody. In some embodiments of any of the embodiments disclosed herein, the kit further comprises c) a secondary antibody. In some embodiments of any of the embodiments disclosed herein, the antibody or the secondary antibody is conjugated to an enzyme. In some embodiments of any of the embodiments disclosed herein, the kit further comprises d) a substrate. In some embodiments of any of the embodiments disclosed herein, cmvIL-10 is LAcmvIL-10.

In further aspects, provided herein are methods for detecting human cytomegalovirus (HCMV) in a biological sample provided by an individual, the method comprising: (a) contacting the biological sample comprising viral interleukin 10 (cmvIL-10) with a probe that specifically binds to a cmvIL-10 polypeptide or nucleic acid; and (b) detecting the presence of cmvIL-10 when a complex is formed between the probe and cmvIL-10 polypeptide or nucleic acid, wherein the individual is seronegative for HCMV and wherein the individual has been diagnosed with breast cancer. In some embodiments, the probe comprises one or more nucleic acids. In some embodiments, the one or more nucleic acids specifically hybridize to a nucleic acid of SEQ ID NO:1 or SEQ ID NO: 2. In some embodiments of any of the embodiments disclosed herein, said one or more nucleic acids are PCR primers and PCR is performed subsequent to the complex forming between the PCR primers and the cmvIL-10 nucleic acid. In some embodiments of any of the embodiments disclosed herein, the one or more nucleic acids is detectably labeled. In some embodiments, the probe comprises an antibody or fragment thereof. In some embodiments, the antibody or fragment thereof is a monoclonal antibody. In some embodiments, the antibody is a polyclonal antibody. In some embodiments, the polyclonal antibody is produced using a recombinantly-produced cmvIL-10 polypeptide immunogen comprising A26 to K176 of SEQ ID NO:3. In some embodiments of any of the embodiments disclosed herein, the polyclonal antibody is derived from goat. In some embodiments of any of the embodiments disclosed herein, the method further comprises (c) contacting the biological sample with a probe that specifically binds to one or more polypeptides or nucleic acids selected from the group consisting of plasminogen activator inhibitor 1 (PAI-1), urokinase plasminogen activator (uPA), urokinase plasminogen activator receptor (uPAR), matrix metalloproteinase-3 (MMP-3) and missing-in-metastasis (MTSS) and (d) detecting the presence of one or more polypeptides or nucleic acids selected from the group consisting of plasminogen activator inhibitor 1 (PAI-1), urokinase plasminogen activator (uPA), urokinase plasminogen activator receptor (uPAR), matrix metalloproteinase-3 (MMP-3) and missing-in-metastasis (MTSS) when a complex is formed between the probe and the one or more polypeptides or nucleic acids.

In yet other aspects, provided herein is a complex comprising (a) a probe and (b) a cmvIL10 protein or nucleic acid, wherein the cmvIL10 protein or nucleic acid is derived from a biological sample from an individual diagnosed with breast cancer, wherein the individual is infected with human cytomegalovirus (HCMV) but has not undergone seroconversion. In some embodiments, the probe comprises one or more nucleic acids. In some embodiments, the one or more nucleic acids specifically hybridize to a nucleic acid of SEQ ID NO: 1 or SEQ ID NO: 2. The complex of any one of claims 40-41, wherein said one or more nucleic acids are PCR primers and PCR is performed subsequent to the complex forming between the PCR primers and the cmvIL-10 nucleic acid. In some embodiments of any of the embodiments disclosed herein, the one or more nucleic acids is detectably labeled. In some embodiments, the probe comprises an antibody or fragment thereof. In some embodiments, the antibody or fragment thereof comprises a monoclonal antibody. In some embodiments, the antibody comprises a polyclonal antibody. In some embodiments, the polyclonal antibody is produced using a recombinantly-produced cmvIL-10 polypeptide immunogen comprising A26 to K176 of SEQ ID NO:3. In some embodiments of any of the embodiments disclosed herein, the polyclonal antibody is derived from goat.

Each of the aspects and embodiments described herein are capable of being used together, unless excluded either explicitly or clearly from the context of the embodiment or aspect.

### BRIEF DESCRIPTION OF THE DRAWINGS

FIG. 1 depicts human breast cancer cells express the IL-10 receptor. A) MDA-MB-231 cells were stained with anti-IL-10R-PE antibody (black line) or isotype control (gray line) and analyzed by flow cytometry. B) Cells were untreated or treated with 100 ng/ml cmvIL-10 for 15 min and stained for IL-10R followed by TRITC-conjugated secondary antibody, then visualized by fluorescence microscopy. Red corresponds to IL-10R, blue corresponds to DAPI staining of the nucleus. C) RNA was harvested from MDA-MB-231 cells and mock- or HCMV-infected HFF cells (MOI=1, 72 hrs post-infection), reverse-transcribed and IE1 or β-actin gene specific primers were used for PCR. D) MDA-MB-231 and mock- or HCMV-infected HFFs were cultured on glass coverslips, fixed and stained for IE1 followed by FITC-conjugated secondary (green). These results are representative of three independent experiments.

FIG. 2 depicts cmvIL-10 induces Stat3 phosphorylation in human breast cancer cells. A) MDA-MB-231 cells were treated with 100 ng/ml cmvIL-10, hIL-10, or IFNγ for 15 min, then lysed and Western blotted with the indicated antibodies. B) Cells were grown in 96-well dishes and treated with the indicated doses of cmvIL-10 for 15 min before lysis in the well followed by quantification of total vs. pStat3 levels. Results are represented as the normalized ratio of pStat3 to total Stat3 in relative fluorescence units (RFUs). *=p<0.01, Student's t-test. Error bars represent standard error for three replicates of each condition. C) MDA-MB-231 cells were cultured and supernatants collected at the indicated time points were subjected to SDS-PAGE followed by immunoblotting with the indicated antibodies. Control indicates purified recombinant protein (cmvIL-10, hIL-10, or serpin E1/PAI) was loaded as a positive control for each respective antibody. Results are representative of three independent experiments.

FIG. 3 depicts cmvIL-10 stimulates proliferation and increases DNA synthesis in human breast cancer cells. A) MDA-MB-231 cells were grown in 96-well dishes and treated with the indicated doses of cmvIL-10. Cell Titer Glo was added at the indicated time points to measure cell viability, represented as relative light units (RLUs) based on the resulting chemiluminescence. B) Cell growth in the presence or absence of 100 ng/ml cmvIL-10 via Cell Titer Glo. C) BrdU incorporation in the presence of 100 ng/ml cmvIL-10 or hIL-10. D) Standard cell counts of cultures from 6-well dishes containing 100 ng/ml cmvIL-10 or hIL-10 using a hemacytometer. E) BrdU incorporation was assessed at 72 hrs in cells cultured in the presence of 10 ng/ml of each indicated cytokine. F) Cells were treated with 10 μM of each indicated inhibitor or an equivalent volume of DMSO in the presence of absence of 10 ng/ml cmvIL-10 and BrdU incorporation measured after 72 hrs. Error bars represent standard error among three replicates for each condition. * indicates p<0.05, Student's t-test. These results are representative of three independent experiments.

FIG. 4 depicts human breast cancer cells are protected from apoptosis by cmvIL-10. A) MDA-MB-231 cells were treated with 100 μM etoposide in the presence or absence of 100 ng/ml cmvIL-10 for 48 hrs, then stained for Annexin V and analyzed by flow cytometry. B) MDA-MB-231 cells were grown in 96-well dishes and treated with the indicated doses of etoposide in the presence or absence of 100 ng/ml cmvIL-10 for 48 hours, then cell viability evaluated via the addition of Cell Titer Glo and detection of resulting chemiluminescence. C) Cells were cultivated in the presence of 10 μM etoposide with or without 100 ng/ml cmvIL-10. Cell viability was evaluated at the indicated time points via Cell Titer Glo. Error bars represent standard error of three replicates per condition. * indicates p<0.01, Student's t-test. These results are representative of two independent experiments.

FIG. 5 depicts human breast cancer cells exhibit enhanced chemotaxis when exposed to cmvIL-10. MDA-MB-231 cells were seeded at a density of 2×105 cells in a total volume of 0.1 ml in the upper chamber of an 8 μm trans-well filter. A) Complete media containing the indicated concentrations of EGF in the presence or absence of 100 ng/ml cmvIL-10 or hIL-10 was placed in the lower chamber. After 5 hrs, cells in the lower chamber were harvested and quantified by the addition of Cell Titer Glo to measure luminescence. B) Complete medium containing the indicated concentrations of cmvIL-10 in the presence or absence of 1 ng/ml EGF. Cells traversing the filter after 5 hrs were quantified as described. Error bars represent standard error. * indicates p<0.05, Student's t-test. Results are representative of three independent experiments.

FIG. 6 depicts detection of vIL-10 in a representative set of healthy blood donors. Four samples were HCMV seronegative (S9-S12) and four samples were HCMV seropositive (S13-16). Bar graph shows vIL-10 levels as determined by ELISA, and vIL-10 was detected in two seronegative specimens. The seronegative donors with vIL-10 lacked any measurable IgG or IgM response to HCMV (Trinity Bioscience ELISA), but viral DNA could be detected by PCR. A nested PCR procedure was used to detect exon 4 of HCMV IE1 (detailed protocol on next pages) on genomic DNA isolated from the whole blood sample, and top panel represents first round of PCR. Middle panel, second round of PCR shows that two seronegative donors have viral DNA present in their blood, which correlates with detection of vIL-10 protein. Lower panel, detection of 3-actin as a control.

FIG. 7 depicts that cmvIL-10 enhances CXCL12/CXCR4 calcium mobilization and migration. A) HEK293 cells were loaded with Fluo-4 AM calcium indicator dye, then stimulated with CXCL12 (0.1 μg/ml) in the presence or absence of cmvIL-10 (0.1 μg/ml). Relative fluorescence intensity (FLI) was measured over time by flow cytometry and arrow indicates addition of stimulus. B) Calcium flux assay on HEK293 cells showing peak fluorescence intensity with increasing doses of cmvIL-10 in the presence or absence of 0.1 μg/ml CXCL12 or C) increasing doses of CXCL12 in the presence or absence of 0.1 μg/ml cmvIL-10. D) Transwell migration assay with HEK293 cells seeded in a 96-well plate at a density of 5×104 cells in the upper chamber and CXCL12 in the presence or absence of 0.1 μg/ml cmvIL-10 in the lower chamber. After 4 hours, cells that traversed the 5.0 μm filter were harvested and quantified by CellTiter Glo. Error bars represent standard error, * indicates p<0.05, ** p<0.01, *** p<0.001 by paired Student's t-test. These results are representative of three independent experiments.

FIG. 8. depicts that CMV seropositive donors have higher antibody titers. (A) Percent total CMV IgG positive donors in case and control groups. In CMV seropositive women with breast cancer (cases), (B) mean and (C) median IgG ISR (internal standard ratio) values were higher than in the control group.

FIG. 9. depicts that cmvIL-10 and hIL-10 levels are higher in cases vs controls. Donor plasma samples were diluted to 10% in PBS and tested for cmvIL-10 and hIL-10 by ELISA. Results below the lower limit of the assay (15.625 μg/ml) were reported as 0 μg/ml and results above the range of the assay were reported as the upper limit of 1000 μg/ml for the cmvIL-10 or 2000 μg/ml for hIL-10. The average (A) cmvIL-10 and (B) hIL-10 levels were higher in cases compared to controls. Each dot represents a single donor.

FIG. 10. depicts CmvIL-10 and hIL-10 correlations in cases and controls. Levels of cmvIL-10 in plasma correlate more strongly with hIL-10 in (A) cases compared to (B) controls. Each dot represents a single donor.

FIG. 11. depicts that MDA-MB-231 cells exhibit increased migration and matrigel invasion in the presence of cmvIL-10. A) Transwell migration toward EGF after 5 hours in the presence of conditioned medium from mock or HCMV-infected fibroblasts at the indicated MOIs. IL-10R neutralizing antibody (NAb) was included at 30 ug/ml. B) Matrigel invasion toward EGF in the presence of 100 ng/ml purified recombinant cmvIL-10 or hIl-10 after 22 hours. C) Matrigel invasion toward EGF in the presence or absence of 100 ng/ml purified recombinant cmvIL-10 or conditioned medium from mock or infected fibroblasts (MOI=1). Where indicated, 10 uM Stat3 inhibitor was included. Error bars=SEM. * indicates p<0.05, **p<0.001. These results are representative of 3 independent experiments.

FIG. 12. depicts that CmvIL-10 induces changes in metastasis-related gene expression in MDA-MB-231 cells. RNA was extracted from cells treated with 100 ng/ml cmvIL-10 or hIL-10 for 5 hours, then RNA was purified and expression of 84 genes was analyzed using the Human Tumor Metastasis RT2 Profiler PCR Array. A) Select genes encoding extracellular matrix proteins or b) cell adhesion proteins is shown. Fold changes represent comparison to untreated MDA-MB-231 and are the average of three biological replicates. Error bars=SEM. * indicates p<0.05. A complete list of genes analyzed is found in Table 2.

FIG. 13. indicates that PAI-1 and uPAR levels are elevated upon exposure to cmvIL-10 or hIL-10. A) MDA-MB-231 cells were cultivated in the presence of 10 ng/ml purified recombinant cmvIL-10 or hIL-10. At the indicated time points, culture supernatants were collected and levels of PAI-1 measured by ELISA. B) MDA cells were cultured as above and levels of uPAR in the supernatant were determined by ELISA. Error bars represent standard error among 3 replicates for each data point. * indicates p<0.05. These results are representative of 3 independent experiments.

FIG. 14. depicts that MMP-3 expression and activity are increased by cmvIL-10. A) MDA-MB-231 cells were cultured in the presence or absence of 10 ng/ml cmvIL-10 for the indicated times and then cell lysates were analyzed by ELISA. Error bars=SEM, * indicates p<0.05. B) MDA cells cultured with 100 ng/ml cmvIL-10 for the indicated times were harvested and lysates examined by western blotting with anti-MMP3 or anti-MAPK as a protein loading control. Lysates from cells receiving the same treatment were analyzed under non-reducing conditions on a 4-16% Zymogram gel (lower panel). These results are representative of three independent experiments.

FIG. 15. depicts that MTSS1 expression is significantly decreased upon exposure to cmvIL-10. A) MDA-MB-231 cells were cultured with 100 ng/ml cmvIL-10 for the indicated times and lysates examined by western blotting with anti-MTSS1 or anti-β-actin as a protein loading control. B) MDA cells were stained for total surface MTSS1 using anti-MTSS1 followed by PE-conjugated goat-anti rabbit secondary antibody and analyzed by flow cytometry. C) MDA cells were seeded onto glass coverslips in the presence of absence of 100 ng/ml cmvIL-10 for 96 hours, then fixed, permeabilized, and stained as indicated. Scale bar=10 um. These results are representative of three independent experiments.

FIG. 16. shows a model depicting possible role of cmvIL-10 in promoting tumor metastasis. A monocyte that is latently infected with HCMV infiltrates a localized tumor, releasing cmvIL-10 that acts on tumor cells expressing the IL-10 receptor. This leads to changes in levels of MTSS1, uPAR and PAI-1, which reduce adhesion cell adhesion. Increased levels of uPAR and PAI-1 are strongly associated with increased migration and can also help activate MMP-3. Active MMP-3 degrades proteins in the extracellular matrix, facilitating access for tumor cells to invade surrounding stromal tissue and enter the bloodstream.

### DETAILED DESCRIPTION

This invention provides, inter alia, methods for predicting, assessing, or determining the occurrence of metastasis in an individual affected with or thought to have breast cancer as well as methods for determining the risk of metastasis in an individual affected with or thought to have a breast tumor. The invention is based, in part, on the inventors' observations that the human cytomegalovirus (HCMV)-encoded viral interleukin-10 (cmvIL-10) protein can induce changes in human breast cancer cells, leading to tumor metastasis, and that cmvIL-10 is detectable in biological samples derived from individuals who do not possess antibodies to HCMV (i.e. who are seronegative for HCMV) and thus would not be considered to be infected with this virus by the most commonly used laboratory tests employed to screen for the presence of HCMV. As such, the methods disclosed herein provide a valuable tool for clinicians and other health care practitioners for assessing the risk associated with HCMV-mediated breast cancer metastasis in individuals that would not ordinarily be considered at risk due to the lack of HCMV antibodies in their serum.

### I. General Techniques

The practice of the present invention will employ, unless otherwise indicated, conventional techniques of molecular biology, microbiology, cell biology, biochemistry, nucleic acid chemistry, and immunology, which are well known to those skilled in the art. Such techniques are explained fully in the literature, such as, Molecular Cloning: A Laboratory Manual, fourth edition (Sambrook et al., 2012) and Molecular Cloning: A Laboratory Manual, third edition (Sambrook and Russel, 2001), (jointly referred to herein as “Sambrook”); Current Protocols in Molecular Biology (F. M. Ausubel et al., eds., 1987, including supplements through 2014); PCR: The Polymerase Chain Reaction, (Mullis et al., eds., 1994); Antibodies: A Laboratory Manual, Second edition, Cold Spring Harbor Laboratory Press, Cold Spring Harbor, N.Y. (Greenfield, ed., 2014), Beaucage et al. eds., Current Protocols in Nucleic Acid Chemistry, John Wiley & Sons, Inc., New York, 2000, (including supplements through 2014), Gene Transfer and Expression in Mammalian Cells (Makrides, ed., Elsevier Sciences B.V., Amsterdam, 2003), and Current Protocols in Immunology (Horgan K and S. Shaw (1994) (including supplements through 2014).

### II. Definitions

As used herein, “breast cancer” refers to a cancer that starts in a tissue of the breast, such a ductal carcinoma or lobular carcinoma and includes both early stage and late stage breast cancer. Breast cancer may be invasive or non-invasive and/or comprise malignant epithelial cells. Optionally, breast cancer may be classified according to molecular subtypes such as estrogen receptor (ER) and/or Her2 positive or negative as known in the art. In another embodiment, “breast cancer” refers to a cancer that starts in a non-adjacent tissue but which later metastasizes to the breast.

As used herein, “metastasis” refers to the spread of breast cancer from the breast to a non-adjacent part, tissue or organ of the test subject. In one embodiment, metastasis includes “lymph node metastasis” and/or “distant metastasis.” As used herein, “lymph node metastasis” refers to the spread of cancer to the lymph system of a test subject. For example, lymph node metastasis includes the presence of malignant cells in one or more lymph nodes of a test subject, such as in the lymph nodes that are proximal to the breast cancer, for example in one or more sentinel lymph nodes. “Distant metastasis” refers to metastasis that is present in another non-adjacent part, tissue or organ of a test subject such as in lung, liver, brain or bone or in a distal lymph node.

The term “individual” as used herein refers to any member of the animal kingdom, preferably a human being including, for example, a subject having or suspected of having breast cancer. In one embodiment, the subject is a mammal.

“Seroconversion,” as used herein, refers to the point in time when a specific antibody (such as an antibody to HCMV) becomes detectable in a biological sample provided by an individual. During an infection or immunization, antigens enter the blood and the immune system begins to produce antibodies in response. Seroconversion is the point in time when the antibody becomes detectable. Before seroconversion, the antigen may be detectable, but the antibody is not.

As used herein, the phrase “seronegative for HCMV,” means that an individual does not produce any detectable antibodies directed against HCMV (e.g. antibodies detectable by currently available, standard, or routine diagnostic tests). In some embodiments, the phrase refers to a biological sample provided by an individual that lacks any detectable antibodies against HCMV.

“Viral interleukin-10,” or “cmvIL-10,” or “vIL-10” or “cytomegalovirus interleukin-10” can be used interchangeably to refer to the full length mRNA or protein product of the HCMV UL111A gene. In some embodiments, cmvIL-10 also refers to truncated cmvIL-10 (i.e., latency associated cmvIL-10 or LAcmvIL-10).

“Latency Associated cmvIL-10” or “LAcmvIL-10,” as used herein, refers to a truncated mRNA or protein product of the HCMV UL111A gene. The LAcmvIL-10 protein is co-linear with full length cmvIL-10 for the first 127 residues and then diverges in sequence at the truncated C-terminal domain (139 amino acids total compared to 175 for full length cmvIL-10).

As used herein, a “nucleic acid” or “oligonucleotide” refers to two or more deoxyribonucleotides and/or ribonucleotides covalently joined together in either single or double-stranded form.

As used herein, the term “protein” includes polypeptides, peptides, fragments of polypeptides, and fusion polypeptides.

The transitional term “comprising” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Unless defined otherwise herein, all technical and scientific terms used herein have the same meaning as commonly understood by one of ordinary skill in the art to which this invention pertains.

As used herein, the singular terms “a,” “an,” and “the” include the plural reference unless the context clearly indicates otherwise.

### III. Methods of the Invention

In some aspects, provided herein are methods for predicting, assessing, or determining the occurrence of metastasis in an individual affected with breast cancer who is seronegative for HCMV as well as determining the risk of metastasis in an individual affected with a breast tumor that is seronegative for HCMV. The methods encompass detecting the presence of viral interleukin-10 (vIL-10 or cmvIL-10) in a biological sample provided by the individual. The presence of cmvIL-10 indicates that the individual's breast cancer has metastasized or that the individual is at increased risk of tumor metastasis, respectively. In some embodiments, the methods of the present invention optionally include the step of assaying the biological sample from the individual for the presence of antibodies to HCMV to ascertain the individual's HCMV serostatus.

A. Breast Cancer

In the methods provided herein, the individual is affected with or thought to have breast cancer. As used herein, the phrase “an individual affected with breast cancer” or “an individual affected with a breast tumor” means that the individual has or is suspected of having breast cancer. Breast cancer or breast tumors may be diagnosed using any and all available means known in the art. These include, without limitation, physical examination of the breasts by a healthcare provider, mammography, analysis of breast tissue-derived fluid obtained by fine needle aspiration, fine needle aspiration and cytology (FNAC), core biopsy, vacuum-assisted core biopsy, excisional biopsy, magnetic resonance imaging (MRI), or ultrasound.

The methods of the invention can be utilized with any stage of breast cancer (such as, Stage 0, Stage I, Stage II, Stage III, or Stage IV) based on the TNM Classification of Malignant Tumours (TNM).

The methods of the invention are also appropriate for use irrespective of the receptor status or molecular subtype of the tumor. The receptor status of breast cancers has traditionally been identified by immunohistochemistry (IHC), which stains cells based on the presence of estrogen receptors (ER), progesterone receptors (PR) and HER2. This is the most common method of testing for receptor status, but DNA multi-gene expression profiles can categorize breast cancers into molecular subtypes that generally correspond to IHC receptor status. One such commercially available assay for categorizing the molecular subtype of breast cancer is the BluePrint® assay manufactured by Agendia.

Any form of breast cancer can be assessed for metastasis or metastatic risk according to the methods of the present invention. These include, without limitation, invasive ductal carcinomas (e.g., Mixed type carcinoma, Pleomorphic carcinoma, Carcinoma with osteoclast giant cells, Carcinoma with choriocarcinoma features, or Carcinoma with melanotic features), Invasive lobular carcinoma, Tubular carcinoma, Invasive cribriform carcinoma, Medullary carcinoma, Mucinous carcinoma (and other tumors with abundant mucin, such as, Mucinous carcinoma Cystadenocarcinoma and columnar cell mucinous carcinoma, Signet ring cell carcinoma), Neuroendocrine tumors (such as Solid neuroendocrine carcinoma (carcinoid of the breast), Atypical carcinoid tumor, Small cell/oat cell carcinoma Large cell neuroendocrine carcinoma), Invasive papillary carcinoma, Invasive micropapillary carcinoma, Apocrine carcinoma, Metaplastic carcinomas (such as Pure epithelial metaplastic carcinomas (e.g., Squamous cell carcinoma, Adenocarcinoma with spindle cell metaplasia, Adenosquamous carcinomasukers, Mucoepidermoid carcinoma) and Mixed epithelial/mesenchymal metaplastic carcinomas), Matrix-producing carcinoma, Spindle cell carcinoma, Carcinosarcoma, Squamous cell carcinoma of mammary origin, Metaplastic carcinoma with osteoclastic giant cells, Lipid-rich carcinoma, Secretory carcinoma, Oncocytic carcinoma, Adenoid cystic carcinoma, Acinic cell carcinoma, Glycogen-rich clear cell carcinoma, Sebaceous carcinoma, Inflammatory carcinoma, Bilateral breast carcinoma, Mesenchymal tumors (including sarcoma, such as Hemangioma, Angiomatosis, Hemangiopericytoma, Pseudoangiomatous stromal hyperplasia, Myofibroblastoma, Fibromatosis (aggressive), Inflammatory myofibroblastic tumor, Lipoma (e.g., Angiolipoma), Granular cell tumour, Neurofibroma, Schwannoma, Angiosarcoma, Liposarcoma, Rhabdomyosarcoma, Osteosarcoma, Leiomyoma, Leiomyosarcoma), Tumors of the male breast (e.g., Gynecomastia and Carcinoma (In situ and Invasive), Metastatic tumors to the breast from other places in the body, Precursor lesions (such as, Lobular neoplasia, lobular carcinoma in situ, Intraductal proliferative lesions, Usual ductal hyperplasia, Flat epithelial hyperplasia, Atypical ductal hyperplasia, Ductal carcinoma in situ, Microinvasive carcinoma, Intraductal papillary neoplasms, Central papilloma, Peripheral papilloma, Atypical papilloma, Intraductal papillary carcinoma, Intracystic papillary carcinoma), Myoepithelial lesions (such as, Myoepitheliosis, Adenomyoepithelial adenosis, Adenomyoepithelioma, Malignant myoepithelioma), Fibroepithelial tumours, (such as, Fibroadenoma, Phyllodes tumour (Benign, Borderline, or Malignant), Periductal stromal sarcoma (low-grade), Mammary hamartoma or malignant tumors of the nipple (e.g., Paget's disease).

B. Human Cytomegalovirus (HCMV)

Human Cytomegalovirus (HCMV) belongs to the β-Herpesvirinae subfamily of the family Herpesviridae, including human herpes virus 6 (HHV-6) and human herpes virus 7 (HHV-7). HCMV is a double-stranded DNA virus, comprising 230 kbp coding more than 200 genes with the diameter of about 180 nm. HCMV is the biggest virus among the family Herpesviridae. HCMV exhibits strong species specificity as no other animals are known to be vulnerable to HCMV infection. The specific mode of HCMV transmission from person to person is unknown but is presumed to occur through bodily fluids.

After infection, HCMV remains latent in lymphocytes in the body for the rest of the individual's life. Overt disease rarely occurs unless immunity is suppressed either by drugs, infection, or old age. Initial HCMV infection, which often is asymptomatic, is followed by a prolonged, latent infection during which the virus resides in mononuclear cells without causing detectable damage or clinical illness. However, in immunocompromised patients such as hemodialysis patients, cancer patients, patients who take immunosuppressants, HIV-carriers, bone-marrow transplant patients, and organ transplant patients with immunocompromised status, HCMV can be reactivated into an active, lytic infection. Life-threatening diseases such as interstitial pneumonia, retinitis, gastroenteritis, and encephalitis can subsequently develop in these individuals.

Persons who have been infected with HCMV develop antibodies to the virus and, like the HCMV infection, these antibodies persist in the body for the lifetime of that individual. A number of laboratory tests that detect these antibodies to HCMV have been developed to determine if infection has occurred and are widely available from commercial laboratories. The enzyme-linked immunosorbent assay (or ELISA) is the most commonly available serologic test for measuring and/or detecting antibody to HCMV. For example, the result of an ELISA test can be used to determine if acute infection, prior infection, or passively acquired maternal antibody in a newborn infant is present.

Aside from ELISA, another method that can be used to determine if an individual is infected with HCMV is assaying for the presence of a HCMV-derived nucleic acid (such as an RNA or genomic DNA) in sample obtained from the individual. In some embodiments, PCR or RT-PCR is used to determine if an individual is infected with HCMV. In one embodiment, the PCR is performed to detect the presence of HCMV genomic DNA (such as the DNA sequence encoded by Genbank Accession no. X17403 or a portion thereof). In another embodiment, the PCR assays for the presence of the HCMV IE1 gene. In a further embodiment, the PCR assay for the HCMV IE1 gene uses a forward primer having the sequence AAGTGAGTTCTGTCGGGTGCT and a reverse primer having the sequence GTGACACCAGAGAATCAGAGGA.

C. Viral Interleukin-10 (cmvIL-10)

The cmvIL-10 protein is a homolog of human IL-10 encoded by the UL111A gene product of HCMV (Kotenko et al., (2000) Proc Natl Acad Sci USA 97: 1695-1700; Genbank Accession no. X17403 (HCMV AD169 Whole Genome)). Despite having only 27% sequence identity to human IL-10, cmvIL-10 binds to the cellular IL-10 receptor (IL-10R) and displays many of the immune suppressive functions of human IL-10 (Slobedman et al., (2009) J Virol 83; 9618-9629; Spencer et al., (2002) J Virol 76: 1285-1292). These include regulation of IFN-γ, IL-1α, GM-CSF, IL-6 and TNF-α, which are all pro-inflammatory cytokines. CmvIL-10 has also been shown to play a role in downregulating MHC I and MHC II and up regulating HLA-G (non-classical MHC 1). These two events allow for immune evasion by suppressing the cell-mediated immune response and natural killer cell response, respectively.

CmvIL-10 is encoded as a discontinuous open reading frame containing two introns (nucleotides 159678 to 160364 of HCMV AD169 Whole Genome).

(SEQ ID NO: 1)
atg ctgtcggtga tggtctatc ctctctggtc ctgatcgtct
tttttctagg cgcttccgag gaggcgaagc cggcgacgac
gacgataaag aatacaaagc cgcagtgtcg tccagaggat
tacgcgacca gattgcaaga tctccgcgtc acctttcatc
gagtaaaacc tacgttggta ggtcacgtag gtacggttta
ttgcgacggt ctttcttttc cgcgtgtcgg gtgacgtagt
tttcctcttg tagcaacgtg aggacgacta ctccgtgtgg
ctcgacggta cggtggtcaa aggctgttgg ggatgcagcg
tcatggactg gttgttgagg cggtatctgg agatcgtgtt
tcccgcaggc gaccacgtct atcccggact caagacggaa
ttgcatagta tgcgctcgac gctagaatcc atctacaaag
acatgcggca atgtgtaagt gtctctgtgg cggcgctgtc
cgcacagagg taacaacgtg ttcatagcac gctgttttac
ttttgtcggg ctcccagcct ctgttaggtt gcggagataa
gtccgtgatt agtcggctgt ctcaggaggc ggaaaggaaa
tcggataacg gcacgcggaa aggtctcagc gagttggaca
cgttgtttag ccgtctcgaa gagtatctgc actcgagaaa
gtag (introns shown in bold)

The presence of introns in the UL111A gene encoding the cmvIL-10 mRNA allows for the possibility of alternative splicing, and this has been documented to occur in latently infected granulocyte-macrophage progenitor cells (Jenkins et al., (2004) J Virol. 78(3): 1440-7). The truncated UL111A region latency-associated (LAcmvIL-10) transcript differs from full length cmvIL-10 transcripts in that it contains only one intron (nucleotides 159678 to 160173 of HCMV AD169 Whole Genome).

(SEQ ID NO: 1)
tgcggcgatg ctgtcggtga tggtctcttc ctctctggtc
ctgatcgtct tttttctagg cgcttccgag gaggcgaagc
cggcgacgac gacgataaag aatacaaagc cgcagtgtcg
tccagaggat tacgcgacca gattgcaaga tctccgcgtc
acctttcatc gagtaaaacc tacgttggta ggtcacgtag
gtacggttta ttgcgacggt ctttcttttc cgcgtgtcgg
gtgacgtagt tttcctcttg tagcaacgtg aggacgacta
ctccgtgtgg ctcgacggta cggtggtcaa aggctgttgg
ggatgcagcg tcatggactg gttgttgagg cggtatctgg
agatcgtgtt tcccgcaggc gaccacgtct atcccggact
caagacggaa ttgcatagta tgcgctcgac gctagaatcc
atctacaaag acatgcggca atgtgtaagt gtctctgtgg
cggcgctgtc cgcacagagg taa (intron shown
in bold)

The LAcmvIL-10 protein product (SEQ ID NO: 4) is co-linear with cmvIL-10 for the first 127 residues and then diverges in sequence at the truncated C-terminal domain (139 amino acids total compared to 175 for full length cmvIL-10 (SEQ ID NO:3)).

(SEQ ID NO: 3)
MLSVMVSSSL VLIVFFLGAS EEAKPATTTT IKNTKPQCRP
EDYATRLQDL RVTFHRVKPT LQREDDYSVW LDGTVVKGCW
GCSVMDWLLR RYLEIVFPAG DHVYPGLKTE LHSMRSTLES
IYKDMRQCPL LGCGDKSVIS RLSQEAERKS DNGTRKGLSE
LDTLFSRLEE YLHSRK
(SEQ ID NO: 4)
MLSVMVSSSL VLIVFFLGAS EEAKPATTTT IKNTKPQCRP
EDYATRLQDL RVTFHRVKPT LQREDDYSVW LDGTVVKGCW
GCSVMDWLLR RYLEIVEPAG DHVYPGLKTE LHSMRSTLES
IYKDMRQCVS VSVAALSAQR

Whereas full length cmvIL-10 exhibits a broad range of inhibitory functions associated with human IL-10, including inhibition of PBMC proliferation, impairment of dendritic cell maturation expression, suppression of inflammatory cytokine synthesis, and reduction of class II MHC expression, the immunosuppressive activities of LAcmvIL-10 appear to be more attenuated.

The UL111A gene is expressed in both lytic and latent HCMV infection. Both cmvIL-10 and LAcmvIL-10 are expressed during lytic HCMV infection. However, only LAcmvIL-10 has been shown to be produced during latent infection.

The ability to evade recognition from the immune system is essential for cancer cells and HCMV is highly adept at manipulating the host immune system (Scalzo et al., (2007) Immunol Cell Biol 85: 46-54). Interestingly, elevated levels of 1-10 are frequently detected in the serum of cancer patients and correlate with poor clinical outcomes (Llanes-Fernandez L et al., (2006) Breast 15: 482-489; Nicolini et al., (2006) Cytokine Growth Factor Rev 17: 325-337; Asadullah et al., (2003) Pharmacol Rev 55: 241-269; Althwani & Najm (2011) J Fac Med—Baghdad 53: 289-292) suggesting that 1-10 may contribute to immune suppression and protect tumor cells from cytotoxic T lymphocytes by down-regulation of class I and class II MHC. In vitro, IL-10 has been found to promote resistance to apoptosis in human breast and lung cancer cell lines (Zeng et al., (2009) Cytokine; Zeng et al., (2007) Cancer Immunol Immunother 56: 205-215). Furthermore, constitutive activation of Stat3, the primary downstream activator associated with IL-10 signaling, correlates with poor prognosis in ovarian cancer and is considered a key factor in the development of metastasis and resistance to chemotherapeutic agents (Zhang et al., (2010) Cancer Genet Cytogenet 197: 46-53).

D. Biological Samples

Biological samples for use in the methods of the instant invention can be obtained from individuals by any means known in the art and can include, without limitation, blood (including, e.g., products derived from whole blood, such as serum or platelets), tissue, urine, saliva, semen, tears, cerebrospinal fluid, or breast milk.

Biological samples can also be obtained directly from breast tumors in any number of various ways. In one aspect, a biological sample is obtained from a tumor which can be a subcutaneously accessible tumor or from any other type of cancerous solid tumor accessible to biopsy or surgical removal. The biological sample may be obtained by any method known in the art including, but not limited to, needle or core biopsy or fine needle aspiration. Additionally, the biological sample may be fixed, paraffin embedded, fresh, or frozen before expression levels of cmv-IL10 are measured.

E. Detection of cmvIL-10 Expression

Viral interleukin-10 expression or activity can be detected in samples using any means known in the art. CmvIL-10 expression encompasses the existence of the full and intact UL111A viral DNA sequence (including, e.g., promoter elements, enhancer sequences, introns, and exons), the conversion of the UL111A gene sequence into transcribed mRNA (including, e.g., the initial unspliced mRNA transcript or the mature processed mRNA), or the translated cmvIL-10 (or LAcmvIL-10) protein product (including, e.g., any posttranslational modifications such as, but not limited to, ubiquitination, sumoylation, acetylation, methylation, glycosylation, and/or hydroxylation).

1. cmvIL-10 Gene and mRNA Expression

The assessment of cmvIL-10 expression or activity in a sample can be at the levels of mRNA or DNA. Assessment of mRNA expression levels of gene transcripts is routine and well known in the art. For example, one flexible and sensitive quantitative method for assessing mRNA expression levels in a biological sample is by quantitative RT-PCR (qRT-PCR) or by any other comparable quantitative PCR-based method. Additional methods for assessing cmvIL-10 mRNA expression include, but are not limited to, Northern blotting, microarrays, in situ hybridization, and serial analysis of gene expression (SAGE).

Techniques such as Northern blotting or RT-PCR rely on the use of a probe or primers complementary to the cmv-IL10 mRNA. The term “complementary” and “complementarity” refer to polynucleotides (i.e., a sequence of nucleotides) related by the base-pairing rules. For example, the sequence 5′-A-T-G-C-3′ is complementary to the sequence 5′-G-C-A-T-3′. Complementarity may be “partial,” in which case only some of the bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in PCR-based amplification reactions, as well as detection methods that depend upon binding between nucleic acids. In some embodiments, nucleic acid probes such as oligonucleotides, oligonucleotide arrays, and/or primers for use in the methods of the present invention are complementary to a nucleic acid of SEQ ID NO:1 or SEQ ID NO:2. In other embodiments, nucleic acid probes such as oligonucleotides, oligonucleotide arrays, and/or primers for use in the methods of the present invention are any of about 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementary to a nucleic acid of SEQ ID NO:1 or SEQ ID NO:2. In further embodiments, nucleic acid probes such as oligonucleotides, oligonucleotide arrays, and/or primers for use in the methods of the present invention are at least about 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementary to a nucleic acid of SEQ ID NO:1 or SEQ ID NO:2. In some embodiments, nucleic acid probes such as oligonucleotides, oligonucleotide arrays, and/or primers for use in the methods of the present invention are at most about 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementary to a nucleic acid of SEQ ID NO:1 or SEQ ID NO:2. All individual values for numbers and percentages as used herein can be combined to create upper and lower limits for ranges. For example, in additional embodiments, nucleic acid probes such as oligonucleotides, oligonucleotide arrays, and/or primers for use in the methods of the present invention can be about 65%-100%, 75%-95%, 80%-90%, 75%-100%, 80%-100%, 85%-100%, 85%-95%, 90%-100%, or 95%-100% complementary to a nucleic acid of SEQ ID NO:1 or SEQ ID NO:2.

Nucleic acid binding molecules such as probes, oligonucleotides, oligonucleotide arrays, and primers can be used in assays to detect cmvIL-10 or LAcmvIL-10 RNA expression in biological samples from individuals with breast cancer. In one embodiment, RT-PCR is used according to standard methods known in the art. In another embodiment, PCR assays such as Taqman® assays available from, e.g., Applied Biosystems, can be used to detect nucleic acids and variants thereof. In another embodiment, a two stage nested PCT assay (such as that performed in Example 5, infra) is performed to assess the existence of the cmvIL-10 mRNA or genomic DNA sequence. In other embodiments, qPCR and nucleic acid microarrays can be used to detect nucleic acids. Reagents that bind to cmvIL-10 or LAcmvIL-10 can be prepared according to methods known to those of skill in the art or purchased commercially.

Analysis of cmvIL-10 or LAcmvIL-10 nucleic acids can be achieved using routine techniques such as Southern blot analysis, PCR, Northern blot analysis, RT-PCR, or any other methods based on hybridization to a nucleic acid sequence that is complementary to a portion of the cmvIL-10 or LAcmvIL-10 coding sequence (e.g., slot blot hybridization or fluorescence in situ hybridization (FISH)) are also within the scope of the present invention. General nucleic acid hybridization methods are described in Anderson, “Nucleic Acid Hybridization,” BIOS Scientific Publishers, 1999. Amplification or hybridization of a plurality of nucleic acid sequences (e.g., genomic DNA, mRNA or cDNA) can also be performed from mRNA or cDNA sequences arranged in a microarray. Microarray methods are generally described in Hardiman, “Microarrays Methods and Applications: Nuts & Bolts,” DNA Press, 2003; and Baldi et al., “DNA Microarrays and Gene Expression: From Experiments to Data Analysis and Modeling,” Cambridge University Press, 2002.

Analysis of the gene encoding cmvIL-10 (UL111A) can be performed using techniques known in the art including, without limitation, microarrays, polymerase chain reaction (PCR)-based analysis, sequence analysis, and electrophoretic analysis. A non-limiting example of a PCR-based analysis includes a Taqman® allelic discrimination assay available from Applied Biosystems. Non-limiting examples of sequence analysis include Maxam-Gilbert sequencing, Sanger sequencing, capillary array DNA sequencing, thermal cycle sequencing (Sears et al., Biotechniques, 13:626-633 (1992)), solid-phase sequencing (Zimmerman et al., Methods Mol. Cell Biol., 3:39-42 (1992)), sequencing with mass spectrometry such as matrix-assisted laser desorption/ionization time-of-flight mass spectrometry (MALDI-TOF/MS; Fu et al., Nat. Biotechnol., 16:381-384 (1998)), and sequencing by hybridization. Chee et al., Science, 274:610-614 (1996); Drmanac et al., Science, 260:1649-1652 (1993); Drmanac et al., Nat. Biotechnol., 16:54-58 (1998). Non-limiting examples of electrophoretic analysis include slab gel electrophoresis such as agarose or polyacrylamide gel electrophoresis, capillary electrophoresis, and denaturing gradient gel electrophoresis. Other methods for detecting nucleic acids include, e.g., the INVADER® assay from Third Wave Technologies, Inc., restriction fragment length polymorphism (RFLP) analysis, allele-specific oligonucleotide hybridization, a heteroduplex mobility assay, single strand conformational polymorphism (SSCP) analysis, single-nucleotide primer extension (SNUPE) and pyrosequencing.

A detectable moiety or detectable label can be used in the assays described herein for detection of cmvIL-10 nucleic acids. A wide variety of detectable moieties can be used, with the choice of label depending on the sensitivity required, ease of conjugation, stability requirements, and available instrumentation and disposal provisions. Suitable detectable moieties include, but are not limited to, radionuclides, fluorescent dyes (e.g., fluorescein, fluorescein isothiocyanate (FITC), Oregon Green™, rhodamine, Texas red, tetrarhodimine isothiocynate (TRITC), Cy3, Cy5, etc.), fluorescent markers (e.g., green fluorescent protein (GFP), phycoerythrin, etc.), autoquenched fluorescent compounds that are activated by tumor-associated proteases, enzymes (e.g., luciferase, horseradish peroxidase, alkaline phosphatase, etc.), nanoparticles, biotin, digoxigenin, and the like.

Detection of cmvIL-10 nucleic acids can be carried out in a variety of physical formats. For example, the use of microtiter plates or automation could be used to facilitate the processing of large numbers of biological samples. Alternatively, single sample formats could be developed to facilitate diagnosis or prognosis in a timely fashion.

Alternatively, the nucleic acid probes of the invention can be applied to sections of biological sample biopsies immobilized on microscope slides. The resulting staining or in situ hybridization pattern can be visualized using any one of a variety of light or fluorescent microscopic methods known in the art.

Also provided herein are reagents for in vivo imaging of cmvIL-10 (or LAcmvIL-10) such as, for instance, the imaging of labeled regents that detect cmvIL-10 nucleic acids. For in vivo imaging purposes, reagents that detect the presence of cmvIL-10 (or LAcmvIL-10) nucleic acids, may be labeled using an appropriate marker, such as a fluorescent marker.

2. Protein Expression

Similarly, assessment of protein expression levels is routine in the art. For example, one method of measuring protein levels is via Western blotting or immunohistochemistry using antibodies to cmvIL-10 (or LAcmvIL-10). Other well-known and reliable methods for assaying for the existence of cmvIL-10 protein in a sample include, without limitation, radioimmunoassay (RIA), ELISA (such as the ELISA described in Example 5, infra), flow cytometry, immunohistochemistry, immunocytochemistry, or any other antibody-mediated technique.

Antibody reagents can be used in assays to detect expression of cmvIL-10 (or LAcmvIL-10) in patient samples using any of a number of immunoassays known to those skilled in the art. “Antibody” as used herein is meant to include intact molecules as well as fragments which retain the ability to bind antigen, such as cmvIL-10 (e.g., Fab and F(ab′) fragments). These fragments are typically produced by proteolytically cleaving intact antibodies using enzymes such as a papain (to produce Fab fragments) or pepsin (to produce F(ab)2 fragments). The term “antibody” also refers to both monoclonal antibodies and polyclonal antibodies. Polyclonal antibodies are derived from the sera of animals immunized with the antigen.

Antibodies having specificity for a specific protein, such as a protein product of the UL111A gene (such as the proteins encoded by SEQ ID NOs:3-4), mRNA, or a fragment thereof, may be prepared by conventional methods. A mammal, (e.g. a mouse, hamster, or rabbit) can be immunized with an immunogenic form of the peptide which elicits an antibody response in the mammal. Techniques for conferring immunogenicity on a peptide include conjugation to carriers or other techniques well known in the art. For example, the peptide can be administered in the presence of adjuvant. The progress of immunization can be monitored by detection of antibody titers in plasma or serum. Standard ELISA or other immunoassay procedures can be used with the immunogen as antigen to assess the levels of antibodies. Following immunization, antisera can be obtained and, if desired, polyclonal antibodies isolated from the sera.

In some embodiments, a polyclonal antibody that binds to cmvIL-10 and LAcmvIL-10 for use in any of the antibody-based detection methods disclosed herein (e.g., ELISA) is produced using an immunogenic peptide encoding SEQ ID NOs:3 or 4 or a portion of the proteins encoded by SEQ ID NOs:3 or 4. In other embodiments, the polyclonal antibody is produced using an immunogenic peptide having any of about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the proteins encoded by SEQ ID NOs:3 or 4. In other embodiments, the polyclonal antibody is produced using an immunogenic peptide at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the proteins encoded by SEQ ID NOs:3 or 4. In other embodiments, the polyclonal antibody is produced using an immunogenic peptide having at most about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the proteins encoded by SEQ ID NOs:3 or 4. In further embodiments, the polyclonal antibody is produced using an immunogenic peptide about 50%-100%, 50%-90%, 50%-80%, 50%-70%, 50%-60%, 55%-100%, 55%-90%, 55%-80%, 55%-70%, 55%-65%, 60%-100%, 60%-90%, 65%-80%, 65%-75%, 65%-100%, 75%-95%, 80%-90%, 75%-100%, 80%-100%, 85%-100%, 85%-95%, 90%-100%, or 95%-100% identity to the proteins encoded by SEQ ID NOs:3 or 4.

In a further embodiment, the polyclonal antibody is produced using an immunogenic peptide comprising A26 to K176 of SEQ ID NO:3. In another embodiment, the polyclonal antibody is produced in goat. In one embodiment, the immunogenic peptide is recombinantly produced in a bacterial species (such as, but not limited to E coli). In yet another embodiment, the Neutralization Dose (ND₅₀) of the polyclonal antibody is about 0.15-0.9 μg/mL (for example, about 0.15 μg/mL-0.8 μg/mL, 0.15 μg/mL-0.7 μg/mL, 0.15 μg/mL-0.6 μg/mL, 0.15 μg/mL-0.5 μg/mL, 0.15 μg/mL-0.4 μg/mL, 0.2 μg/mL-0.8 μg/mL, 0.3 μg/mL-0.7 μg/mL, 0.4 μg/mL-0.6 μg/mL, 0.5 μg/mL-0.9 μg/mL, 0.6 μg/mL-0.9 μg/mL, or 0.7 μg/mL-0.9 μg/mL), such as any of about 0.15 μg/mL, 0.2 μg/mL, 0.25 μg/mL, 0.3 μg/mL, 0.35 μg/mL, 0.4 μg/mL, 0.45 μg/mL, 0.5 μg/mL, 0.55 μg/mL, 0.6 μg/mL, 0.65 μg/mL, 0.7 μg/mL, 0.75 μg/mL, 0.8 μg/mL, 0.85 μg/mL, or 0.9 μg/mL, inclusive of all values falling in between these concentrations) in the presence of 2 ng/mL recombinant cmvIL-10. In other embodiments, the Neutralization Dose (ND₅₀) of the polyclonal antibody is at least about 0.15 μg/mL, 0.2 μg/mL, 0.25 μg/mL, 0.3 μg/mL, 0.35 μg/mL, 0.4 μg/mL, 0.45 μg/mL, 0.5 μg/mL, 0.55 μg/mL, 0.6 μg/mL, 0.65 μg/mL, 0.7 μg/mL, 0.75 μg/mL, 0.8 μg/mL, 0.85 μg/mL, or 0.9 μg/mL, inclusive of all values falling in between these concentrations) in the presence of 2 ng/mL recombinant cmvIL-10. In some embodiments, the Neutralization Dose (ND₅₀) of the polyclonal antibody is at most about 0.15 μg/mL, 0.2 μg/mL, 0.25 μg/mL, 0.3 μg/mL, 0.35 μg/mL, 0.4 μg/mL, 0.45 μg/mL, 0.5 μg/mL, 0.55 μg/mL, 0.6 μg/mL, 0.65 μg/mL, 0.7 μg/mL, 0.75 μg/mL, 0.8 μg/mL, 0.85 μg/mL, or 0.9 μg/mL, inclusive of all values falling in between these concentrations) in the presence of 2 ng/mL recombinant cmvIL-10.

Monoclonal antibodies can be prepared using hybridoma technology (Kohler, et al., Nature 256:495 (1975)). In general, this technology involves immunizing an animal, usually a mouse. The splenocytes of the immunized animals are extracted and fused with suitable myeloma cells, e.g., SP2O cells. After fusion, the resulting hybridoma cells are selectively maintained in a culture medium and then cloned by limiting dilution (Wands, et al., Gastroenterology 80:225-232 (1981)). The cells obtained through such selection are then assayed to identify clones which secrete antibodies capable of binding to septin family member proteins or fragments thereof. Such techniques are well known in the art, (e.g. the hybridoma technique originally developed by Kohler and Milstein (Nature 256:495-497 (1975)) as well as other techniques such as the human B-cell hybridoma technique (Kozbor et al., Immunol. Today 4:72 (1983)), the EBV-hybridoma technique to produce human monoclonal antibodies (Cole et al., Methods Enzymol, 121: 140-67 (1986)), and screening of combinatorial antibody libraries (Huse et al., Science 246:1275 (1989)). Hybridoma cells can be screened immunochemically for production of antibodies specifically reactive with the peptide and the monoclonal antibodies can be isolated.

Immunoassay techniques and protocols using antibodies or fragments thereof to cmvIL-10 or LAcmvIL-10 are generally described in Price and Newman, “Principles and Practice of Immunoassay,” 2nd Edition, Grove's Dictionaries, 1997; and Gosling, “Immunoassays: A Practical Approach,” Oxford University Press, 2000. A variety of immunoassay techniques, including competitive and non-competitive immunoassays, can be used. See, e.g., Self et al., Curr. Opin. Biotechnol., 7:60-65 (1996). The term immunoassay encompasses techniques including, without limitation, enzyme immunoassays (EIA) such as enzyme multiplied immunoassay technique (EMIT), enzyme-linked immunosorbent assay (ELISA), IgM antibody capture ELISA (MAC ELISA), and microparticle enzyme immunoassay (MEIA); capillary electrophoresis immunoassays (CEIA); radioimmunoassays (RIA); immunoradiometric assays (IRMA); fluorescence polarization immunoassays (FPIA); and chemiluminescence assays (CL). If desired, such immunoassays can be automated. Immunoassays can also be used in conjunction with laser induced fluorescence. See, e.g., Schmalzing et al., Electrophoresis, 18:2184-93 (1997); Bao, J. Chromatogr. B. Biomed. Sci., 699:463-80 (1997). Liposome immunoassays, such as flow-injection liposome immunoassays and liposome immunosensors, are also suitable for use in the present invention. See, e.g., Rongen et al., J. Immunol. Methods, 204:105-133 (1997). In addition, nephelometry assays, in which the formation of protein/antibody complexes results in increased light scatter that is converted to a peak rate signal as a function of the marker concentration, are suitable for use in the methods of the present invention. Nephelometry assays are commercially available from Beckman Coulter (Brea, Calif.; Kit #449430) and can be performed using a Behring Nephelometer Analyzer (Fink et al., J. Clin. Chem. Clin. Biochem., 27:261-276 (1989)).

Specific immunological binding of the antibody to cmvIL-10 or LAcmvIL-10 can be detected directly or indirectly. Direct labels include fluorescent or luminescent tags, metals, dyes, radionuclides, and the like, attached to the antibody. An antibody labeled with iodine-125 (¹²⁵I) can be used. A chemiluminescence assay using a chemiluminescent secondary antibody specific for the antibody to cmvIL-10 or LAcmvIL-10 is suitable for sensitive, non-radioactive detection of protein levels. A secondary antibody labeled with a fluorochrome is also suitable. Examples of fluorochromes include, without limitation, DAPI, fluorescein, Hoechst 33258, R-phycocyanin, B-phycoerythrin, R-phycoerythrin, rhodamine, Texas red, and lissamine. Indirect labels include various enzymes well known in the art, such as horseradish peroxidase (HRP), alkaline phosphatase (AP), β-galactosidase, urease, and the like. A horseradish-peroxidase detection system can be used, for example, with the chromogenic substrate tetramethylbenzidine (TMB), which yields a soluble product in the presence of hydrogen peroxide that is detectable at 450 nm. An alkaline phosphatase detection system can be used with the chromogenic substrate p-nitrophenyl phosphate, for example, which yields a soluble product readily detectable at 405 nm. Similarly, a β-galactosidase detection system can be used with the chromogenic substrate o-nitrophenyl-β-D-galactopyranoside (ONPG), which yields a soluble product detectable at 410 nm. A urease detection system can be used with a substrate such as urea-bromocresol purple (Sigma Immunochemicals; St. Louis, Mo.).

A signal from the direct or indirect label can be analyzed, for example, using a spectrophotometer to detect color from a chromogenic substrate; a radiation counter to detect radiation such as a gamma counter for detection of ¹²⁵I; or a fluorometer to detect fluorescence in the presence of light of a certain wavelength. For detection of enzyme-linked antibodies, a quantitative analysis can be made using a spectrophotometer such as an EMAX Microplate Reader (Molecular Devices; Menlo Park, Calif.) in accordance with the manufacturer's instructions. If desired, the assays of the present invention can be automated or performed robotically, and the signal from multiple samples can be detected simultaneously.

In some embodiments, the antibodies can be immobilized onto a variety of solid supports, such as magnetic or chromatographic matrix particles, the surface of an assay plate (e.g., microtiter wells), pieces of a solid substrate material or membrane (e.g., plastic, nylon, paper, nitrocellulose), and the like. An assay strip can be prepared by coating the antibody or a plurality of antibodies in an array on a solid support. This strip can then be dipped into the test sample and processed quickly through washes and detection steps to generate a measurable signal, such as a colored spot.

In some embodiments, the presence of at least about 5 pg/mL of cmvIL-10 protein in the biological sample indicates the cancer has metastasized. In another embodiment, the presence of at least about any of 6 pg/mL, 7 pg/mL, 8 pg/mL, 9 pg/mL, 10 pg/mL, 11 pg/mL, 12 pg/mL, 13 pg/mL, 14 pg/mL, 15 pg/mL, 16 pg/mL, 17 pg/mL, 18 pg/mL, 19 pg/mL, 20 pg/mL, 21 pg/mL, 22 pg/mL, 23 pg/mL, 24 pg/mL, 25 pg/mL, 26 pg/mL, 27 pg/mL, 28 pg/mL, 29 pg/mL, 30 pg/mL, 31 pg/mL, 32 pg/mL, 33 pg/mL, 34 pg/mL, 35 pg/mL, 36 pg/mL, 37 pg/mL, 38 pg/mL, 39 pg/mL, 40 pg/mL, 41 pg/mL, 42 pg/mL, 43 pg/mL, 44 pg/mL, 45 pg/mL, 46 pg/mL, 47 pg/mL, 48 pg/mL, 49 pg/mL, 50 pg/mL, 100 pg/mL, 150 pg/mL, 200 pg/mL, 250 pg/mL, 300 pg/mL, 350 pg/mL, 400 pg/mL, 450 pg/mL, 500 pg/mL, 600 pg/mL, 700 pg/mL, 800 pg/mL, 900 pg/mL, 1 ng/mL, 2 ng/mL, 3 ng/mL, 4 ng/mL, 5 ng/mL, 6 ng/mL, 7 ng/mL, 8 ng/mL, 9 ng/mL, 10 ng/mL, or more, inclusive of concentrations falling within these values, of cmvIL-10 protein in the biological sample indicates the cancer has metastasized. In another embodiment, the presence of at least about any of about 2 pg/mL-7 pg/mL, 5 pg/mL-10 pg/mL, 8 pg/mL-13 pg/mL, 11 pg/mL-16 pg/mL, 14 pg/mL-19 pg/mL, 17 pg/mL-22 pg/mL, 20 pg/mL-25 pg/mL, 1 pg/mL-10 pg/mL, 5 pg/mL-15 pg/mL, 10 pg/mL-30 pg/mL, 1 pg/mL-50 pg/mL, 1 pg/mL-100 pg/mL, 1 pg/mL-200 pg/mL, 1 pg/mL-300 pg/mL, 1 pg/mL-400 pg/mL, 1 pg/mL-500 pg/mL, 1 pg/mL-600 pg/mL, 1 pg/mL-700 pg/mL, 1 pg/mL-800 pg/mL, 1 pg/mL-900 pg/mL, 1 pg/mL-1 ng/mL, or 1 pg/mL-10 ng/mL of cmvIL-10 protein in the biological sample indicates the cancer has metastasized.

In other embodiments, the presence of at least about 5 pg/mL of cmvIL-10 protein in the biological sample indicates that the individual is at increased risk for breast cancer metastasis. In another embodiment, the presence of at least about any of 6 pg/mL, 7 pg/mL, 8 pg/mL, 9 pg/mL, 10 pg/mL, 11 pg/mL, 12 pg/mL, 13 pg/mL, 14 pg/mL, 15 pg/mL, 16 pg/mL, 17 pg/mL, 18 pg/mL, 19 pg/mL, 20 pg/mL, 21 pg/mL, 22 pg/mL, 23 pg/mL, 24 pg/mL, 25 pg/mL, 26 pg/mL, 27 pg/mL, 28 pg/mL, 29 pg/mL, 30 pg/mL, 31 pg/mL, 32 pg/mL, 33 pg/mL, 34 pg/mL, 35 pg/mL, 36 pg/mL, 37 pg/mL, 38 pg/mL, 39 pg/mL, 40 pg/mL, 41 pg/mL, 42 pg/mL, 43 pg/mL, 44 pg/mL, 45 pg/mL, 46 pg/mL, 47 pg/mL, 48 pg/mL, 49 pg/mL, 50 pg/mL, 100 pg/mL, 150 pg/mL, 200 pg/mL, 250 pg/mL, 300 pg/mL, 350 pg/mL, 400 pg/mL, 450 pg/mL, 500 pg/mL, 600 pg/mL, 700 pg/mL, 800 pg/mL, 900 pg/mL, 1 ng/mL, 2 ng/mL, 3 ng/mL, 4 ng/mL, 5 ng/mL, 6 ng/mL, 7 ng/mL, 8 ng/mL, 9 ng/mL, 10 ng/mL, or more, inclusive of concentrations falling within these values, of cmvIL-10 protein in the biological sample indicates that the individual is at increased risk for breast cancer metastasis. In another embodiment, the presence of at least about any of about 2 pg/mL-7 pg/mL, 5 pg/mL-10 pg/mL, 8 pg/mL-13 pg/mL, 11 pg/mL-16 pg/mL, 14 pg/mL-19 pg/mL, 17 pg/mL-22 pg/mL, 20 pg/mL-25 pg/mL, 1 pg/mL-10 pg/mL, 5 pg/mL-15 pg/mL, 10 pg/mL-30 pg/mL, 1 pg/mL-50 pg/mL, 1 pg/mL-100 pg/mL, 1 pg/mL-200 pg/mL, 1 pg/mL-300 pg/mL, 1 pg/mL-400 pg/mL, 1 pg/mL-500 pg/mL, 1 pg/mL-600 pg/mL, 1 pg/mL-700 pg/mL, 1 pg/mL-800 pg/mL, 1 pg/mL-900 pg/mL, 1 pg/mL-1 ng/mL, or 1 pg/mL-10 ng/mL of cmvIL-10 protein in the biological sample indicates that the individual is at increased risk for breast cancer metastasis.

F. Seroconversion in Individuals Infected with HCMV

As discussed above, some embodiments of the disclosed methods include the additional step of assaying the biological sample from the individual for the presence of antibodies to HCMV to determine that individual's serostatus for HCMV.

Persons who have been infected with HCMV develop antibodies to the virus and these antibodies persist in the body for the lifetime of that individual. A number of laboratory tests that detect these antibodies to HCMV have been developed to determine if infection has occurred and are widely available from commercial laboratories (e.g, the Human CMV IgM ELISA antibody test from Human Diagnostics, Inc. and the Captia™ CMV IgG ELISA from Trinity Biotech). Additionally, HCMV assays are also part of the standard screening for non-directed blood donation (i.e., donations not specified for a particular patient) in the U.S., the UK and many other countries throughout the world. The enzyme-linked immunosorbent assay (or ELISA) is the most commonly available serologic test for measuring antibody to HCMV. For example, the result of an ELISA test can be used to determine if acute infection, prior infection, or passively acquired maternal antibody in a newborn infant is present.

G. Gene Expression Dysregulation Induced by cmvIL-10

CmvIL-10 can induce changes in human breast cancer cells, leading to tumor metastasis. Some of these changes are detectable on a gene expression level. Exposure to cmvIL-10 may lead to transcriptional alterations in breast cancer cells, including, for example, altered expression of genes involved in metastasis. In some embodiments, detection of these altered expression levels may be used in screening and/or treatment of breast cancer. In some embodiments, screening of metastasis genes can be in conjunction with screening for cmvIL-10.

In some embodiments, cmvIL-10 can result in upregulation of genes (e.g. plasminogen activator inhibitor 1 (PAI-1), urokinase plasminogen activator (uPA), urokinase plasminogen activator receptor (uPAR), or matrix metalloproteinase-3 (MMP-3)). In some embodiments, cmvIL-10 may result in downregulation of genes (e.g. downregulation of missing-in-metastasis (MTSS)).

In some embodiments, changes in gene expression of one or more of PAI-1, uPA, uPAR, MMP-3, or MTSS in breast cancer cells can comprise a fold upregulation or downregulation of any of about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 4.0, 4.5, 5.0 or more compared to the level of gene expression of one or more of PAI-1, uPA, uPAR, MMP-3, or MTSS in normal breast tissue. In some embodiments, changes in gene expression one or more of PAI-1, uPA, uPAR, MMP-3, or MTSS in breast cancer cells can comprise a fold upregulation or downregulation of about 0.5-5.0, about 1.0-4.5, about 1.5-4.0, about 2.0-3.5, about 2.5-3.0, about 0.5-1.0, about 1.0-1.5, about 1.5-2.0, about 2.0-2.5, about 2.5-3.0, about 3.0-3.5, about 3.5-4.0, about 4.0-4.5, or about 4.5-5.0 compared to the level of gene expression of one or more of PAI-1, uPA, uPAR, MMP-3, or MTSS in normal breast tissue. In some embodiments, changes in gene expression of one or more of PAI-1, uPA, uPAR, MMP-3, or MTSS in breast cancer cells may comprise a percent upregulation or downregulation of about 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%. 440%, 450%, 500% or more compared to the level of gene expression of one or more of PAI-1, uPA, uPAR, MMP-3, or MTSS in normal breast tissue. In some embodiments, changes in gene expression of one or more of PAI-1, uPA, uPAR, MMP-3, or MTSS in breast cancer cells may comprise a percent upregulation or downregulation of about 50%-500%, about 100%-450%, about 150%-400%, about 200%-350%, about 250%-300%, about 50-100%, about 100-150%, about 150-200%, about 200-250%, about 250-300%, about 300-350%, about 350-400%, about 400-450%, about 450%-500% or more compared to the level of gene expression of one or more of PAI-1, uPA, uPAR, MMP-3, or MTSS in normal breast tissue.

H. cmvIL-10 Induction of Calcium Mobilization

cmvIL-10 can amplify CXCR4-mediated calcium signaling in a dose-dependent manner. cmvIL-10 also significantly enhances chemotaxis toward CXCL12. Exposure to cmvIL-10 may lead to alterations in breast cancer cells, including detectable alterations in chemotaxis and calcium mobilization.

In some embodiments, cmvIL-10 may result in increased chemotaxis toward CXCL12. In some embodiments, cmvIL-10 may result in increased CXCR4-mediated calcium signaling (such as an increase of any of about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 4.0, 4.5, 5.0 or more compared to CXCR4-mediated calcium signaling in normal breast tissue).

### IV. Compositions of the Invention

Also provided herein are complexes that include at least a probe and a cmvIL10 (or LAcmvIL-10) protein or nucleic acid, wherein the cmvIL10 protein or nucleic acid is derived from a biological sample from an individual diagnosed with breast cancer or suspected of having breast cancer, wherein the individual is infected with human cytomegalovirus (HCMV) but has not undergone seroconversion. The probe can be any of the nucleic acid or polypeptide probes described herein (such as an antibody or functional fragment thereof). Additionally, the biological sample (such as, blood) from which the cmvIL10 protein or nucleic acid is derived can be processed, prior to formation of the complex, such as by isolation of serum, total protein, or nucleic acids. The complex can be formed by contacting the probe with cmvIL10, for example, ex vivo by using biological samples from the individual having or suspected of having breast cancer.

### V. Kits

In addition, the present invention includes a kit for carrying out the subject cmvIL-10 (or LAcmvIL-10) assays. The kit can include one or more probes specific for identification of cmvIL-10 (and/or LAcmvIL-10) mRNA or protein in a biological sample from an individual. Such probes include can include antibodies (either polyclonal, monoclonal, or fragments thereof) or oligonucleotide probes. The probes can optionally include a signal such as a radioactive isotope (such as, but not limited to, ³H, ¹⁴C, ²H, ¹²⁵I, ³²P, or ³³P), a signal-producing enzyme (such as, for example, horseradish peroxidase, luciferase), or a signal-producing protein (e.g., but not limited to, green fluorescent protein). Where the probe is an antibody or fragment thereof, the kit can also include a secondary antibody conjugated to a signal. In other embodiments, the probe can be an aptamer, photoaptamer, protein, peptide, peptidomimetic or a small molecule chemical compound.

Other materials useful for performing the subject method can also be included as part of the kit. For example, the kit can include buffers or labware necessary to obtain or store a biological sample from an individual, or isolate or purify target mRNA or protein from the biological sample. Further, the kit can include materials (e.g., chemicals or buffers or substrates for eliciting signals from a signal-producing enzyme) or labware for performing hybridization and detection procedures. The kit can also include labeling materials for labeling the probes. Written materials describing the steps involved in the subject method can be included for instructing the user how to use the article of manufacture or kit.

In one embodiment, cmvIL-10 (and/or LAcmvIL-10) proteins derived from a sample can be immobilized on a solid phase or support. The kits may therefore also include reagents and means for measuring the quantity of cmvIL-10 proteins, or fragments thereof. For example, the kits can employ immunoassays, mass spectrometry analysis technology, or chromatographic technology, or a combination of said technologies.

In one embodiment, the kit comprises antibodies, antigen-binding, or complementary nucleic acids for cmvIL-10 (and/or LAcmvIL-10). The kit may comprise probes or assays for detecting expression of mRNA, cDNA or protein corresponding cmvIL-10 (and/or LAcmvIL-10). Suitable probes or assays may include complementary nucleic acids (including cDNA or oligonucleotides, for example) or antibodies, fragments thereof, or antigen-binding polypeptides directed against (i.e. capable of binding) the corresponding cmvIL-10 (and/or LAcmvIL-10) proteins.

The kit may include instructions for use in detecting breast cancer, determining risk of metastasis, determining tumor grade, and determining tumor sub-type. In a specific example, the kit may be useful in predicting metastatic potential of a breast cancer tumor.

In another embodiment, the kit contains reagents necessary for performing an ELISA in accordance with the methods of the present invention (such as the ELISA performed in Example 5, infra). Such kits can include, without limitation, polyclonal or monoclonal antibodies to cmvIL-10 (or LAcmvIL-10), purified cmvIL-10 protein, a secondary antibody (e.g., a biotinylated secondary antibody such as a Biotinylated Affinity Purified Polyclonal Goat IgG antibody), wash buffer, blocking buffer, a signal molecule (such as, but not limited to Streptavidin-HRP), substrate solution, and/or stop solution (such as sulfuric acid). In some embodiments, the ELISA is sensitive to only cmvIL-10 (or LAcmvIL-10) and does not cross react with other human cytokines (such as, without limitation, hIL-10, ebvIL-10, or IFN-γ). In another embodiment, the ELISA reagents contained in the kit can detect at least about 5 pg/mL of cmvIL-10 protein in the sample.

In some embodiments, the presence of at least about 5 pg/mL of cmvIL-10 protein in the biological sample indicates the cancer has metastasized. In another embodiment, the presence of at least about any of 6 pg/mL, 7 pg/mL, 8 pg/mL, 9 pg/mL, 10 pg/mL, 11 pg/mL, 12 pg/mL, 13 pg/mL, 14 pg/mL, 15 pg/mL, 16 pg/mL, 17 pg/mL, 18 pg/mL, 19 pg/mL, 20 pg/mL, 21 pg/mL, 22 pg/mL, 23 pg/mL, 24 pg/mL, 25 pg/mL, 26 pg/mL, 27 pg/mL, 28 pg/mL, 29 pg/mL, 30 pg/mL, 31 pg/mL, 32 pg/mL, 33 pg/mL, 34 pg/mL, 35 pg/mL, 36 pg/mL, 37 pg/mL, 38 pg/mL, 39 pg/mL, 40 pg/mL, 41 pg/mL, 42 pg/mL, 43 pg/mL, 44 pg/mL, 45 pg/mL, 46 pg/mL, 47 pg/mL, 48 pg/mL, 49 pg/mL, 50 pg/mL, 100 pg/mL, 150 pg/mL, 200 pg/mL, 250 pg/mL, 300 pg/mL, 350 pg/mL, 400 pg/mL, 450 pg/mL, 500 pg/mL, 600 pg/mL, 700 pg/mL, 800 pg/mL, 900 pg/mL, 1 ng/mL, 2 ng/mL, 3 ng/mL, 4 ng/mL, 5 ng/mL, 6 ng/mL, 7 ng/mL, 8 ng/mL, 9 ng/mL, 10 ng/mL, or more, inclusive of concentrations falling within these values, of cmvIL-10 protein in the biological sample indicates the cancer has metastasized. In another embodiment, the presence of at least about any of about 2 pg/mL-7 pg/mL, 5 pg/mL-10 pg/mL, 8 pg/mL-13 pg/mL, 11 pg/mL-16 pg/mL, 14 pg/mL-19 pg/mL, 17 pg/mL-22 pg/mL, 20 pg/mL-25 pg/mL, 1 pg/mL-10 pg/mL, 5 pg/mL-15 pg/mL, 10 pg/mL-30 pg/mL, 1 pg/mL-50 pg/mL, 1 pg/mL-100 pg/mL, 1 pg/mL-200 pg/mL, 1 pg/mL-300 pg/mL, 1 pg/mL-400 pg/mL, 1 pg/mL-500 pg/mL, 1 pg/mL-600 pg/mL, 1 pg/mL-700 pg/mL, 1 pg/mL-800 pg/mL, 1 pg/mL-900 pg/mL, 1 pg/mL-1 ng/mL, or 1 pg/mL-10 ng/mL of cmvIL-10 protein in the biological sample indicates the cancer has metastasized.

In other embodiments, the presence of at least about 5 pg/mL of cmvIL-10 protein in the biological sample indicates that the individual is at increased risk for breast cancer metastasis. In another embodiment, the presence of at least about any of 6 pg/mL, 7 pg/mL, 8 pg/mL, 9 pg/mL, 10 pg/mL, 11 pg/mL, 12 pg/mL, 13 pg/mL, 14 pg/mL, 15 pg/mL, 16 pg/mL, 17 pg/mL, 18 pg/mL, 19 pg/mL, 20 pg/mL, 21 pg/mL, 22 pg/mL, 23 pg/mL, 24 pg/mL, 25 pg/mL, 26 pg/mL, 27 pg/mL, 28 pg/mL, 29 pg/mL, 30 pg/mL, 31 pg/mL, 32 pg/mL, 33 pg/mL, 34 pg/mL, 35 pg/mL, 36 pg/mL, 37 pg/mL, 38 pg/mL, 39 pg/mL, 40 pg/mL, 41 pg/mL, 42 pg/mL, 43 pg/mL, 44 pg/mL, 45 pg/mL, 46 pg/mL, 47 pg/mL, 48 pg/mL, 49 pg/mL, 50 pg/mL, 100 pg/mL, 150 pg/mL, 200 pg/mL, 250 pg/mL, 300 pg/mL, 350 pg/mL, 400 pg/mL, 450 pg/mL, 500 pg/mL, 600 pg/mL, 700 pg/mL, 800 pg/mL, 900 pg/mL, 1 ng/mL, 2 ng/mL, 3 ng/mL, 4 ng/mL, 5 ng/mL, 6 ng/mL, 7 ng/mL, 8 ng/mL, 9 ng/mL, 10 ng/mL, or more, inclusive of concentrations falling within these values, of cmvIL-10 protein in the biological sample indicates that the individual is at increased risk for breast cancer metastasis. In another embodiment, the presence of at least about any of about 2 pg/mL-7 pg/mL, 5 pg/mL-10 pg/mL, 8 pg/mL-13 pg/mL, 11 pg/mL-16 pg/mL, 14 pg/mL-19 pg/mL, 17 pg/mL-22 pg/mL, 20 pg/mL-25 pg/mL, 1 pg/mL-10 pg/mL, 5 pg/mL-15 pg/mL, 10 pg/mL-30 pg/mL, 1 pg/mL-50 pg/mL, 1 pg/mL-100 pg/mL, 1 pg/mL-200 pg/mL, 1 pg/mL-300 pg/mL, 1 pg/mL-400 pg/mL, 1 pg/mL-500 pg/mL, 1 pg/mL-600 pg/mL, 1 pg/mL-700 pg/mL, 1 pg/mL-800 pg/mL, 1 pg/mL-900 pg/mL, 1 pg/mL-1 ng/mL, or 1 pg/mL-10 ng/mL of cmvIL-10 protein in the biological sample indicates that the individual is at increased risk for breast cancer metastasis.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The invention can be further understood by reference to the following examples, which are provided by way of illustration and are not meant to be limiting.

### Examples

### Example 1: Uninfected Tumor Cells Express the IL-10R Complex and have the Ability to Respond to cmvIL-10 in the Tumor Microenvironment

In order to determine whether cmvIL-10 could have an impact on tumor cell physiology, we first examined whether breast cancer cells expressed the IL-10R. The MDA-MB-231 breast adenocarcinoma cell line was stained with antibody directed against the alpha chain of the human IL-10R complex and examined via flow cytometry.

Materials and Methods:

Cells & Reagents:

MDA-MB-231 human breast cancer cells (American Type Culture Collection, Manassas, Va.) were cultured in L-15 Leibovitz's Medium (Corning, Manassas, Va.) supplemented with 10% fetal bovine serum (Atlanta Biologicals, Flowery Branch, Ga.) and maintained at 37° C. with atmospheric CO2 according to the suppliers instructions. Purified recombinant cmvIL-10, hIL-10, IFNγ, IL-6 and anti-cmvIL-10, anti-hIL-10, anti-IL-10R, and anti-serpin E1/PAI antibodies were purchased from R&D Systems (Minneapolis, Minn.). Total Stat3, pStat3 (Y705), total Stat1, and pStat1 (Y701) antibodies were from Cell Signaling Technology (Danvers, Mass.). The Stat3 inhibitor was from Santa Cruz Biotechnology (Dallas, Tex.), the Jak1 and p38 MAPK inhibitors were from Calbiochem/EMD Millipore (Billerica, Mass.). Etoposide was from Cayman Chemicals (Ann Arbor, Mich.) and purified recombinant human EGF was from Peprotech (Rocky Hill, N.J.). The HCMV strain AD169 (ATCC) was propagated in human foreskin fibroblasts (HFF, also from ATCC), maintained in Dulbecco's modification of Eagle's medium (Corning) containing 15% fetal bovine serum.

Flow Cytometry:

Monolayer cultures of MDA-MB-231 cells were harvested via gentle scraping according to manufacturer's instructions (R&D Systems), pelleted with centrifugation at 1000×g, and then resuspended in FACS buffer (PBS+1% BSA+0.1% NaN3) at a density of 4.0×106 cells/ml. For each experimental condition, 100 ul of cell were placed into 96-well plates and stained on ice protected from light with phycoerythrin (PE)-conjugated anti-hIL-10α or goat IgG PE isotype control antibody. After 1 hr, the cells were washed three times, resuspended in FACS buffer, and then fixed with 2% paraformaldehyde solution. Cell suspensions were analyzed using a FACSCalibur and CellQuestPro software (BD Biosciences, San Jose, Calif.).

Reverse Transcriptase-Polymerase Chain Reaction:

RNA was isolated from MDA-MB-231, HFF, and HCMV AD169-infected HFF cells using the RNeasy Mini Kit (Qiagen, Valencia, Calif.) according to manufacturer's instructions, followed by cDNA synthesis using the iScript cDNA Synthesis Kit (Bio-Rad, Hercules, Calif.). Each PCR reaction contained cDNA template, primers, dNTP mix, Ex-Taq buffer, and Ex-Taq polymerase (Clontech, Mountain View, Calif.). The gene specific primers for IE1 were 5′-GTGAGTCCGAGGAGATGAAATG-3′ (forward) and 5′-CTCGTAGATAGGCAGCATGAAC-3′ (reverse) and for J-actin 5′-AAGAGAGGCATCCTCACC-3′ (forward) and 5′-TACATGGCTGGGGTGTTG-3′ (reverse). The reaction underwent the following protocol on a T100 Thermal Cycler (Bio-Rad): 94° C. for 5 min followed by 35 cycles of 94° C. for 30 sec, 61° C. for 30 sec, 68° C. for 30 sec, followed by 1 cycle of 68° C. for 5 min, and a final hold at 4° C. The PCR products were visualized on a 3% agarose gel.

Immunofluorescence Microscopy:

MDA-MB-231 cells were seeded into 6-well dishes containing FBS-coated glass coverslips at a density of 2×105 cells per well and then incubated for 48 hrs at 37° C. Cell monolayers were washed with PBS, fixed with 4% paraformaldehyde, then permeabilized with 0.2% (w/v) Triton X-100 followed by treatment with ice cold 50% methanol-50% acetone for 30 min. Cells were then blocked with PBS+10% FBS for 1 hr at 37° C. and stained with anti-IL-10Rα antibody (Santa Cruz Biotechnology) at a 1:100 dilution for 1 hr at 37° C. Following three PBS washes, the coverslips were incubated with TRITC-conjugated secondary antibody for 1 hr, washed again, and then mounted on a glass slide using Prolong Gold anti-fade reagent with DAPI (Life Technologies, Grand Island, N.Y.). HFF cells were also grown on coverslips as described, mock- or virus-infected, and stained with anti-IE1 (EMD Millipore) followed by FITC-conjugated secondary antibody. Images were acquired using a Zeiss LSM700 laser scanning confocal microscope using Zen Black software (Carl Zeiss, Inc., Oberkochen, Germany).

Results:

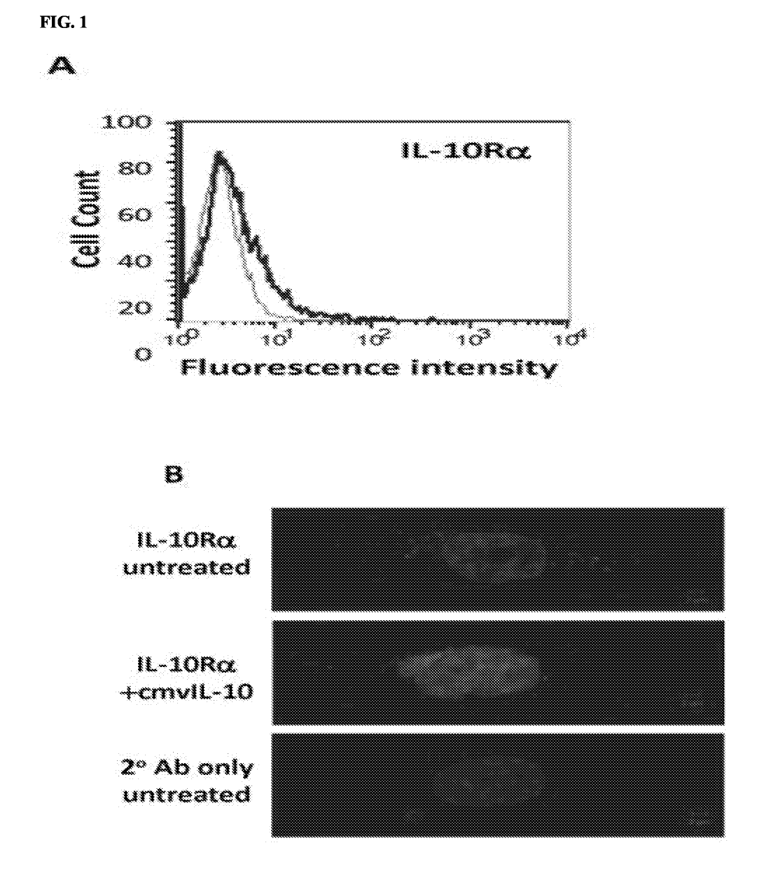

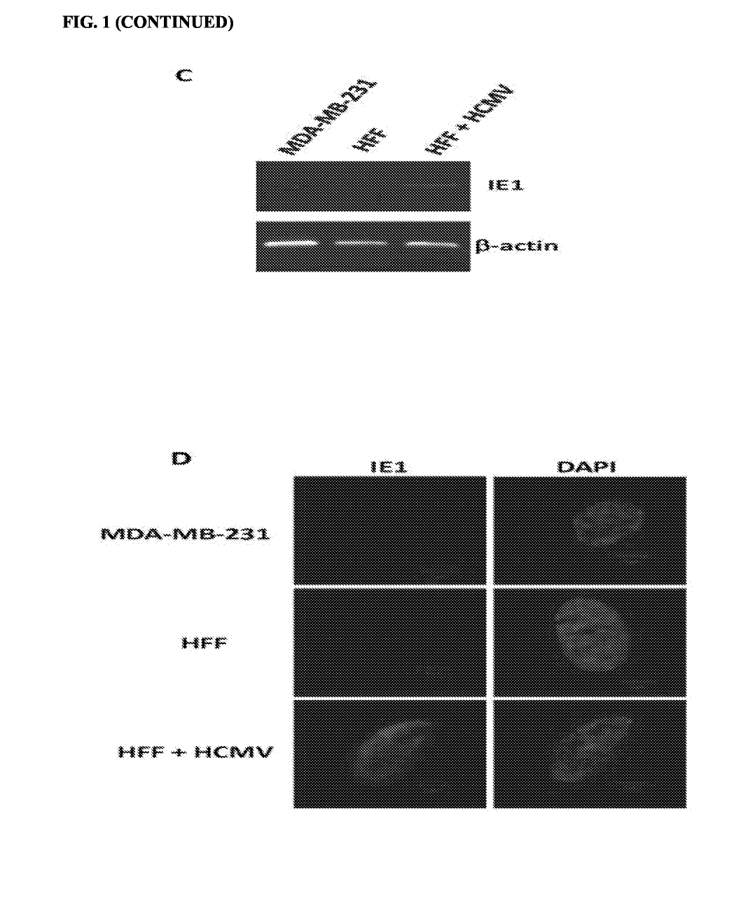

As shown in FIG. 1A, there was low-level expression of the IL-10R complex detected on the surface of these cells. To study receptor distribution in greater detail, the cells were grown on glass cover slips, permeabilized, and visualized with immunofluorescence microscopy. The findings were consistent with the flow cytometry results in that the IL-10R complex was detected on the cell surface (FIG. 1B). However, additional receptor was also observed throughout the inside of the cell, suggesting that the IL-10R complex undergoes constitutive recycling in breast cancer cells and that surface levels are likely to be variable. After treatment with purified recombinant cmvIL-10, there was a distinct redistribution of IL-10R (FIG. 1B), indicating receptor internalization occurred rapidly after ligand engagement. There was no evidence that the MDA-MB-231 breast cancer cell line was infected. Expression of the IE1 gene product could not be detected in these cells by RT-PCR (FIG. 1C) or immunofluorescence staining (FIG. 1D). Human foreskin fibroblasts that were infected with the AD169 strain of HCMV served as a positive control for IE1 expression, which was found to be localized predominantly to the nucleus, as expected (FIG. 1D). These results demonstrated that uninfected tumor cells express the IL-10R complex and have the ability to respond to cmvIL-10 in the tumor microenvironment.

### Example 2: cmvIL-10 can Trigger Phosphorylation and Activation of the Transcription Factor Stat3 in Human Breast Cancer Cells

One of the earliest indicators of cmvIL-10 signaling is phosphorylation of Stat3 by the receptor-associated kinase JAK1. In this example, MDA-MB-231 cells were treated with either cmvIL-10, human IL-10 (hIL-10) or interferon-gamma (IFNγ) and then examined for Stat3 activation.

Materials and Methods:

Western Blots:

For western blotting, cells were treated with 100 ng/ml cmvIL-10, hIL-10, IFNγ or PBS for 15 min, then harvested into cell lysis buffer (150 mM NaCl, 20 mM HEPES, 0.5% Triton-X-100, 1 mM NaOV₄, 1 mM EDTA, 0.1% NaN₃). Lysates were clarified, proteins were separated via SDS-PAGE, and then transferred to a nitrocellulose membrane. Then membrane was incubated in blocking solution (5% milk+1×TBS-T) for 1 hr, then probed with primary antibody at a 1:1000 dilution (total Stat3 or pStat3, total Stat1 or pStat1) in blocking solution overnight at 4° C. After washing, the membranes were incubated with a 1:2000 dilution of appropriate AP-conjugated secondary antibody and bands were detected using Western Blue stabilized AP substrate (Promega, Madison, Wis.). For analysis of secreted proteins, supernatants were collected at various time points, analyzed by SDS-PAGE and then immunoblotted as above for cmvIL-10, hIL-10, or serpin E1/PAI.

Stat 3 ELISA:

For the Stat3 ELISA, cells were seeded into 96-well dishes at a density of 1×10⁴ cell per well, treated with varying doses of cmvIL-10 in triplicate for 15 min, and then lysed in the plate and assayed for total or pStat3 using the Cell-based Stat3 ELISA kit according to manufacturer's instructions (R&D Systems). The detection of hIL-10 in supernatants from MDA-MB-231 cell cultures was performed using the IL-10 ELISA DuoSet kit as directed (R&D Systems).

Results:

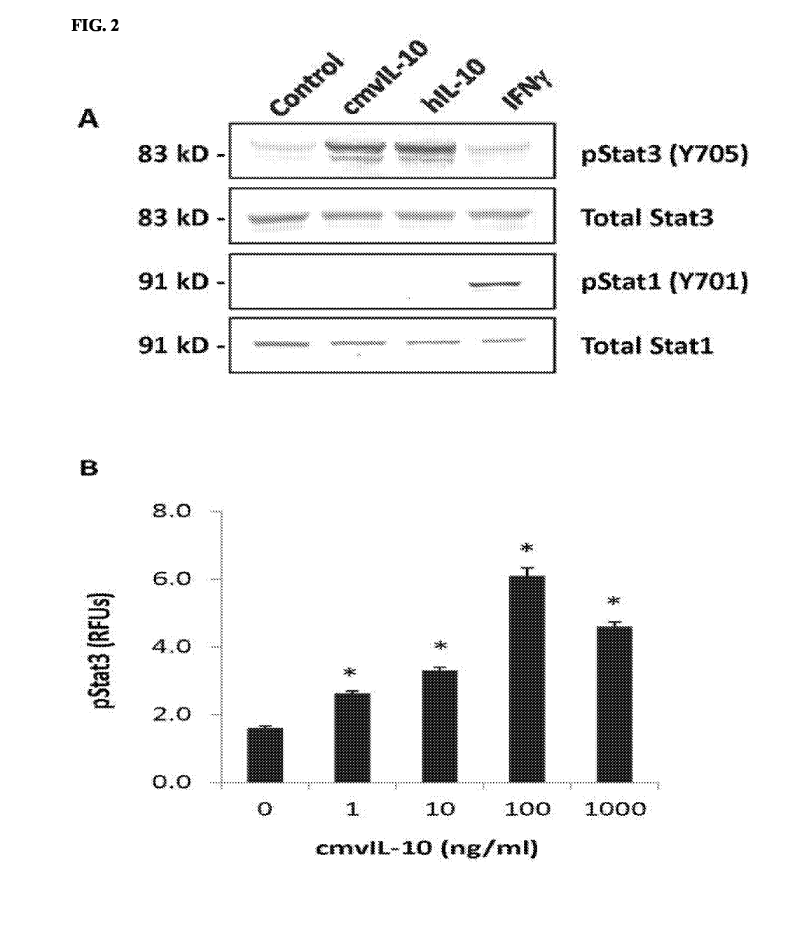

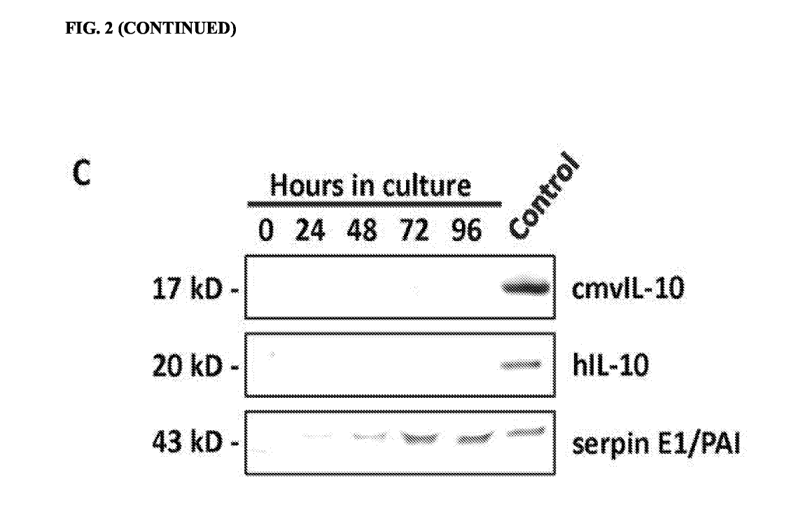

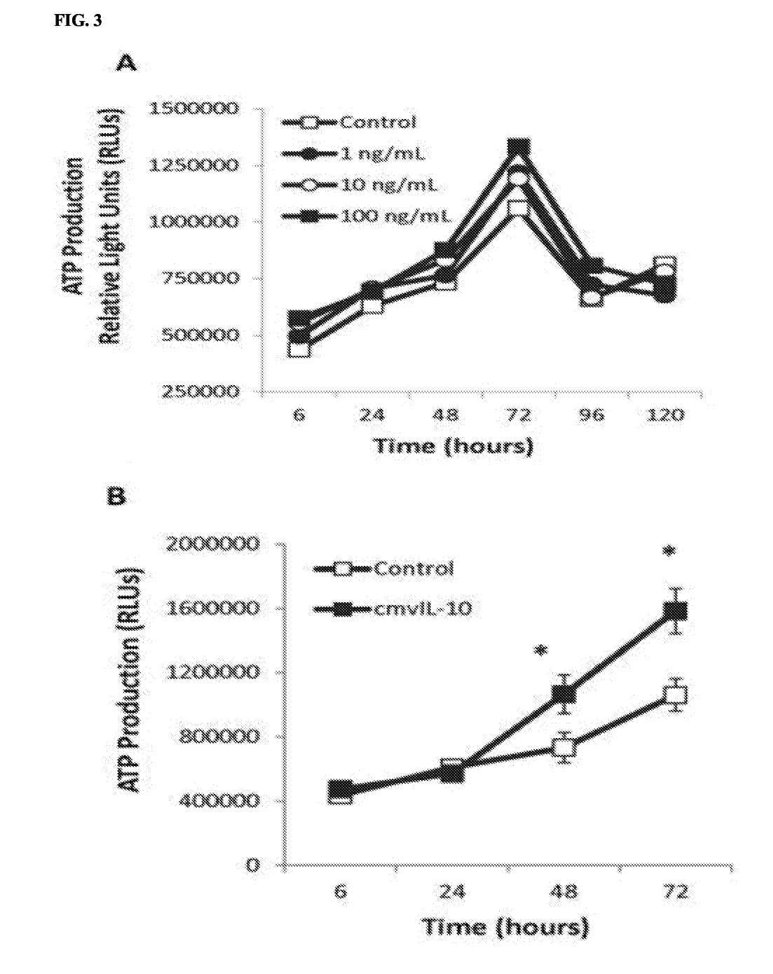

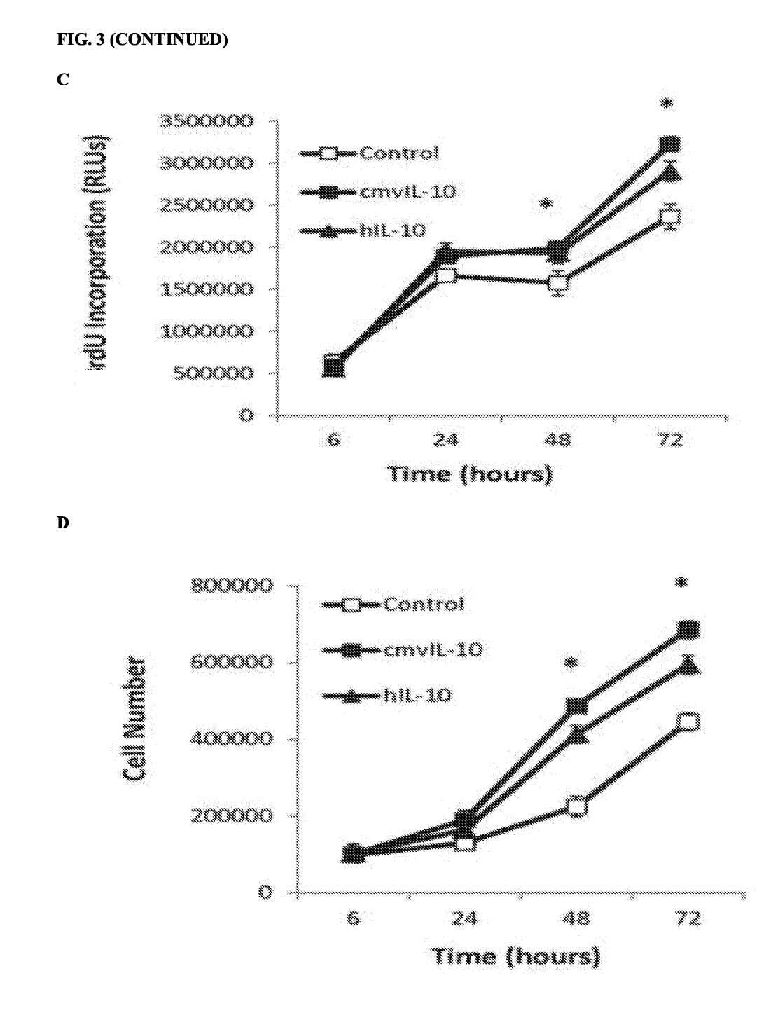

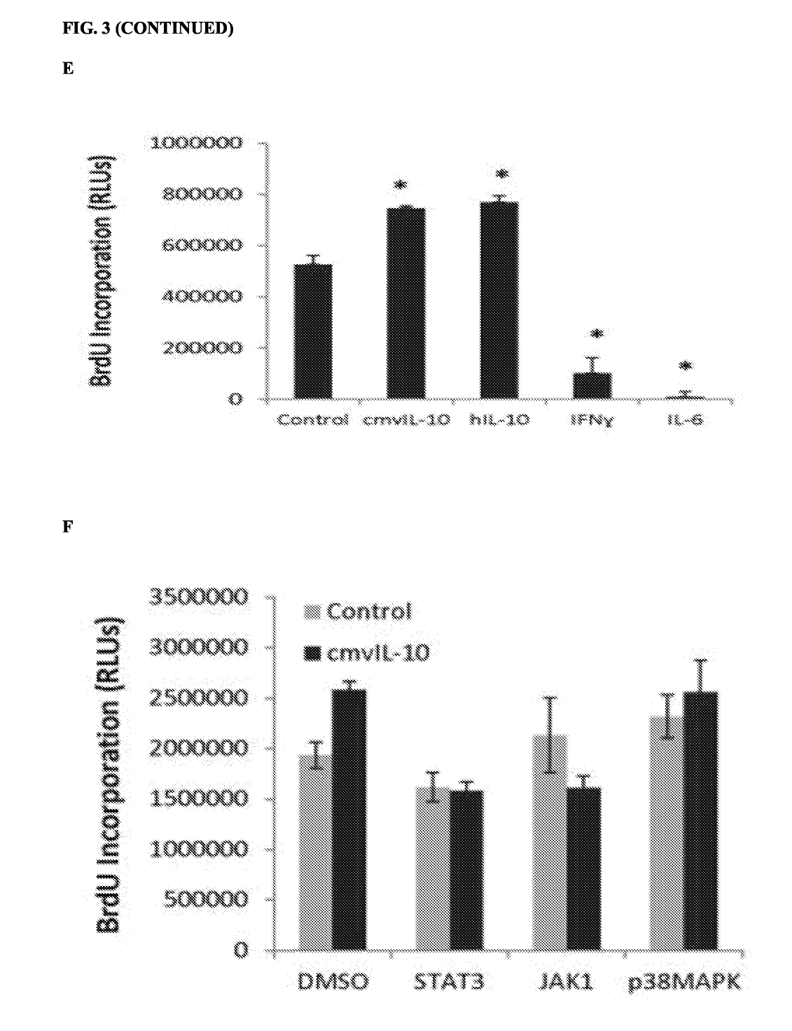

MDA-MB-231 cell lysates were examined by Western blot following treatment with either cmvIL-10, human IL-10 (hIL-10) or interferon-gamma (IFNγ) (FIG. 2A). The expected 83 kD band corresponding to phosphorylated Stat3 (pStat3) was detected in cells treated with cmvIL-10 or hIL-10, but not in control cells exposed to PBS or IFNγ. Exposure to cmvIL-10 specifically activated Stat3, but did not globally activate other cellular effectors, such as Stat1, which was phosphorylated in response to IFNγ treatment only. To confirm Stat3 activation, cells were treated with varying doses of cmvIL-10 and then a cell-based ELISA was performed to detect pStat3. The amount of Stat3 phosphorylation increased in a dose-dependent manner with higher concentrations of cmvIL-10, as shown in FIG. 2B. To confirm that Stat3 activation was solely due to treatment with cmvIL-10, MDA-MB-231 cells were examined for the production of endogenous hIL-10 by both ELISA (data not shown) and Western blot (FIG. 3C). No hIL-10 could be detected in the cell supernatants, confirming that Stat3 was not being activated by an autocrine signaling mechanism. Likewise, cmvIL-10 could not be detected in the supernatants of MDA-MB-231 cells (FIG. 1C), a result that was expected because we found that these cells were not infected with HCMV (FIGS. 1C and D). Human serpin E1 (also known as PAI, plasminogen activator inhibitor-1), which is secreted from many cancer cells, served as a positive control and was detected in cell supernatants in increasing concentrations over time. Taken together, these results demonstrate that exogenous cmvIL-10 can trigger phosphorylation and activation of the transcription factor Stat3 in human breast cancer cells. Over-activation of Stat3 has been documented in glioblastoma, ovarian and breast cancers, which suggests that stimulation of this signaling pathway by cmvIL-10 could contribute to malignancy.

### Example 3: cmvIL-10 Stimulates Cell Proliferation and Increases the Rate of DNA Synthesis in Human Breast Cancer Cells

To elucidate downstream effects of cmvIL-10 signaling and Stat3 activation, cell proliferation was examined in this Example.

Materials and Methods:

Cell Proliferation and Apoptosis Assays:

Cells were seeded into 96-well dishes at a density of 1×10⁴ cell per well in complete medium with varying doses of cmvIL-10, and then cell viability measured at the indicated time points using the Cell Titer Glo Assay kit according to manufacturer's instructions (Promega). DNA synthesis was measured in cells prepared in the same way using the BrdU Cell Proliferation ELISA Kit (Roche, Basel, Switzerland). For experiments using inhibitors, cells were cultivated in 96-well dishes as above with a final concentration of 10 μM inhibitor and BrdU incorporation evaluated after 72 hrs. For cell counts, cells were seeded into 6-well plates at a density of 2×10⁵ cells per well, harvested via trypsinization, and counted using a hemacytometer at the indicated time points. The TACS Annexin V-FITC Detection Kit (Trevigen, Gaithersburg, Md.) was used to stain cells harvested from 70% confluent T75 flasks that had been treated with 100 μM etoposide for 48 hrs in the presence or absence of 100 ng/ml cmvIL-10. Cells were then analyzed via flow cytometry to detect fluorescence. For viability assays, Cell Titer Glo reagent was utilized to quantify cells that had been seeded into 96-well dishes at a density of 1×10⁴ cell per well in complete medium with varying doses of etoposide in the presence or absence of 100 ng/ml cmvIL-10 as indicated

Results:

MDA-MB-231 breast cancer cells were cultured in the presence of increasing doses of cmvIL-10, and cell growth was evaluated. Cell viability was measured by the addition of a luciferin substrate at the indicated time points, and the resulting luminescence is proportional to the amount of ATP present, reflecting the number of viable cells in the well. As shown in FIG. 3A, cells exposed to cmvIL-10 exhibited greater growth than control cells. Overall cell growth increased for 72 hrs and then fell, possibly due to crowding in the wells. Subsequent assays utilized 100 ng/ml cmvIL-10 for 72 hrs, which resulted in significantly higher cell growth than control cultures (FIG. 3B, *=p<0.05). In addition, BrdU incorporation was used to quantify the rate of DNA synthesis, which was found to be significantly higher in cells exposed to cmvIL-10 compared to the control cell lines (FIG. 3C). The level of proliferation induced by cmvIL-10 was comparable to that of hIL-10. Standard cell counts taken at each time point also revealed that cultures treated with either cmvIL-10 or hIL-10 had higher cell numbers than control cultures (FIG. 3D). The enhanced proliferative effect was specific to cmvIL-10 and hIL-10, as treatment with other cytokines did not increase cell proliferation. As shown in FIG. 3E, treatment with IFNγ or IL-6 actually inhibited cell growth. Finally, treatment of cells with either a Stat3 or Jak1 inhibitor blocked the proliferative effects of cmvIL-10, confirming that these results are mediated in part by the Jak1/Stat3 signaling cascade. These results clearly demonstrate that cmvIL-10 specifically stimulates cell proliferation and increases the rate of DNA synthesis in human breast cancer cells.

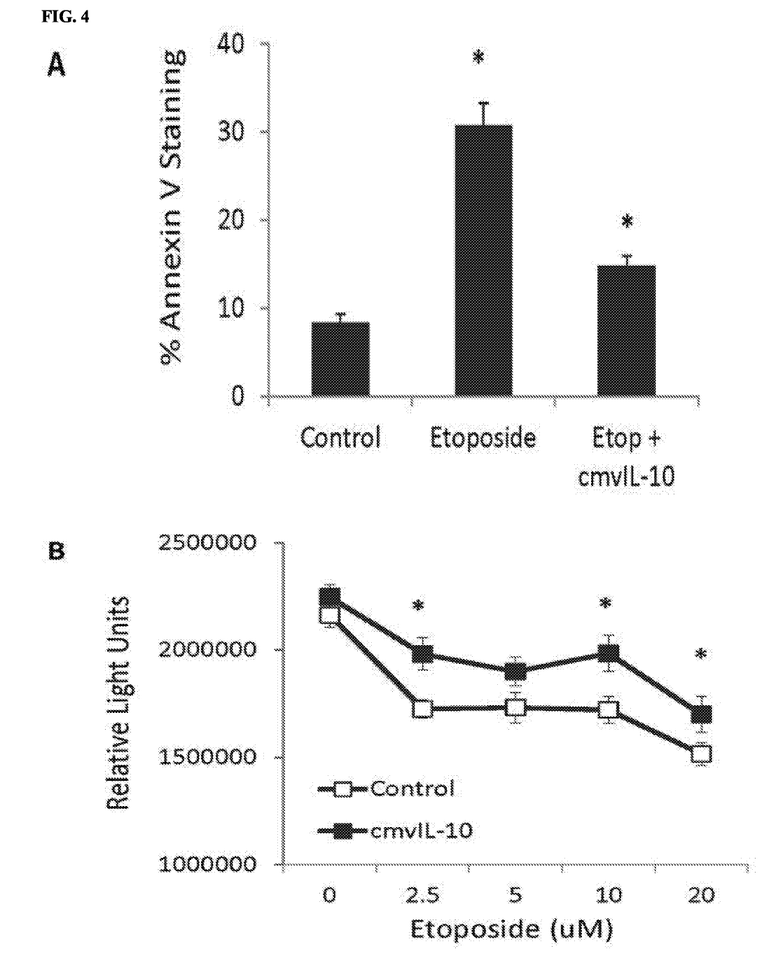

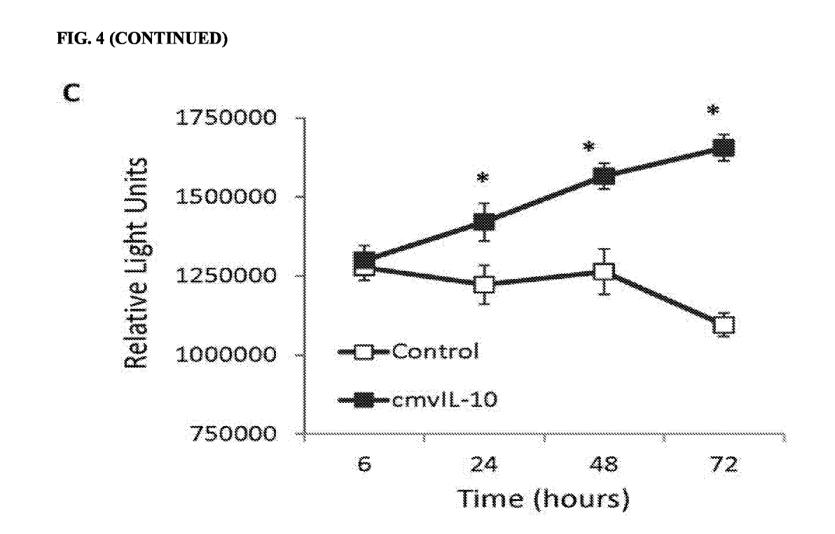

To investigate whether cmvIL-10 could protect cells from apoptosis, MDA-MB-231 breast cancer cells were treated with etoposide, an inhibitor of topoisomerase II that is widely used in the treatment of cancer based on its ability to induce cell death. After exposure to etoposide, 30.9% of cells stained positive for Annexin V via flow cytometry, as shown in FIG. 4A. In contrast, when cultures were incubated with cmvIL-10 prior to etoposide treatment, only 14.8% of cells stained positive for Annexin V, indicating that cmvIL-10 was able to prevent induction of apoptosis in human breast cancer cells. Cultivation of cells with varying doses of etoposide revealed that cmvIL-10 increased overall cell viability (FIG. 4B), and cells exposed to cmvIL-10 were able to overcome etoposide-induced effects over time and proliferate robustly (FIG. 4C).

### Example 4: cmvIL-10 can Work Synergistically with Other Growth Factors and Mitogens Present in the Tumor Microenvironment, to Promote Cell Motility

Because the migration of cancer cells away from the primary tumor is one of the critical early factors in the formation of metastasis, cell motility was investigated in this Example.

Materials and Methods:

Migration Assays:

Cells were harvested and resuspended at a density of 2×10⁶ cells per ml in complete medium. A total volume of 0.1 ml cell suspension (2×10⁵ cells) was placed in the upper chamber of a ThinCert filter with 8 μm pores in a 24-well plate (Greiner Bio-One North America, Monroe, Calif.). A total volume of 0.6 ml of media plus the indicated concentrations of human EGF and/or cmvIL-10 or hIL-10 was added to the lower chamber of each well, and plates were incubated for 5 hrs at 37° C. Medium from the lower chamber was collected, used to rinse the bottom of the filter twice, and then centrifuged at 1000 rpm for 10 min. The cell pellet was resuspended in 0.1 ml media and transferred to a white 96-well plate. Viable cell number was quantified using the Cell Titer Glo Assay kit according to the manufacturer's protocol.

Results:

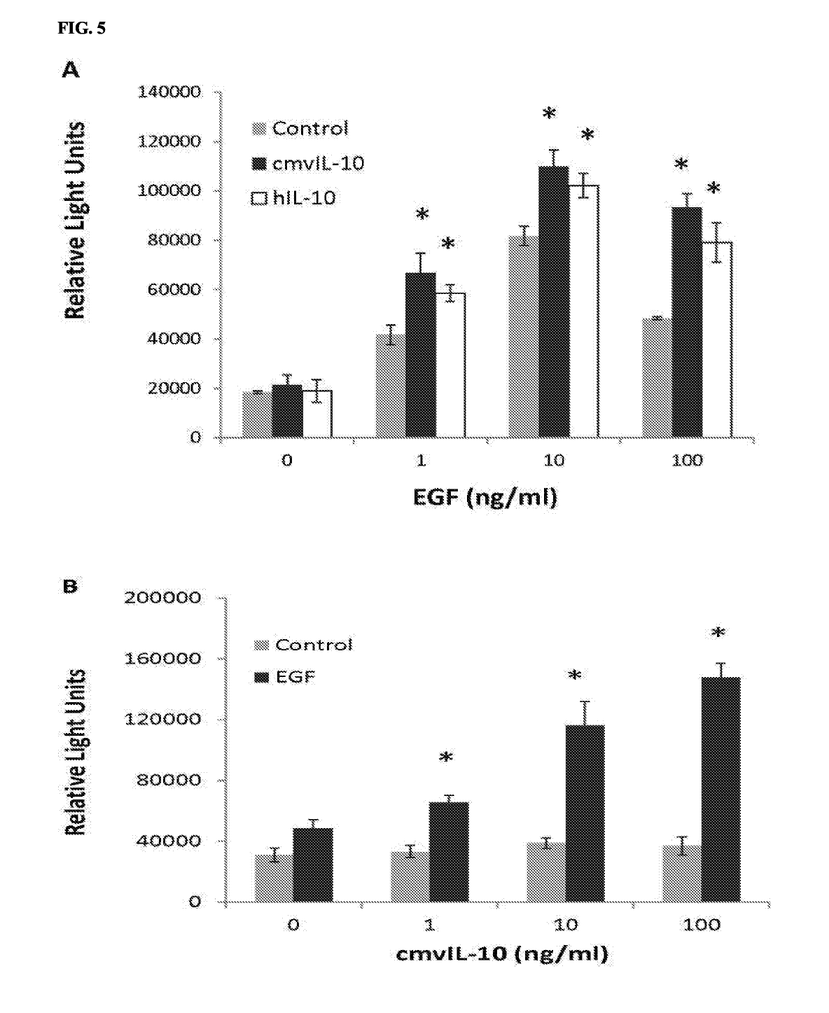

MDA-MB-231 breast cancer cells express the epidermal growth factor (EGF) receptor; therefore, EGF was utilized as a chemo-attractant in a modified Boyden chamber assay. The cells were placed in the top chamber separated from the EGF in the lower chamber by a porous (8 μm) filter, and after 5 hrs cells that had traversed the filter into the lower chamber were harvested and quantified. The cells migrated toward EGF and exhibited a standard bell-shaped curve for chemotaxis with a maximal response at 10 ng/ml EGF (FIG. 5A, gray bars). When both EGF and cmvIL-10 were present in the lower chamber, the migration response was significantly increased (FIG. 5A, black bars), and the effect of cmvIL-10 was comparable to that of hIL-10 (FIG. 5A, white bars). cmvIL-10 alone did not stimulate cell movement (FIG. 5B, gray bars), however, exposure to the viral cytokine significantly enhanced cell migration toward EGF (FIG. 5B, black bars). Exposure to hIL-10 alone also failed to stimulate cell movement (data not shown). These results demonstrate that cmvIL-10 can work synergistically with other growth factors and mitogens present in the tumor microenvironment, such as EGF, to promote increased cell movement.

### Example 5: Detection of vIL-10 in a Representative Set of Healthy Blood Donors

This example demonstrates that individuals who are seronegative for HCMV antibodies can produce vIL-10 at measurable levels in healthy adults and that seronegative individuals may still harbor HCMV.

Materials and Methods:

cmvIL-10 ELISA:

Viral HCMV IL-10 Affinity Purified Polyclonal Ab, Goat IgG (R&D #AF117) was reconstituted in 500 μl of sterile PBS for a final concentration of 200 μg/ml. This was aliquoted into 20 tubes with 25 μl each and store at −20° C. Recombinant viral HCMV IL-10 (R&D #117-VL-025) was reconstituted in 250 μl of sterile PBS containing 0.1% bovine serum albumin (BSA) for a final concentration at 100 μg/ml and aliquoted into 25 tubes with 10 μl each and store at −20° C. Viral HCMV IL-10 Biotinylated Affinity Purified Pab, Goat IgG (R&D #BAF117) was reconstituted in 250 μl of sterile PBS for a final concentration of 200 μg/ml and aliquoted into 25 tubes with 10 μl each and store at −20° C.

Plates were coated with 50 μl/well of 2 μg/ml viral HCMV IL-10 Affinity Purified Polyclonal Ab, Goat IgG (R&D #AF117) diluted in PBS and sealed with adhesive plate cover. The plate was incubated overnight at 4° C. The plate was washed 3× with wash buffer (PBS+0.05% Tween) followed by addition of blocking buffer (PBS+1% BSA), sealing with adhesive, and incubation for 1 hour at room temperature (RT) on a shaker. Following incubation, plates were washed 3× with wash buffer. Samples and standards were then added to the plate and sealed with adhesive cover for a two hour incubation at RT on a shaker. For standards, an 8-point standard curve starting at 1000 μg/ml in PBS containing 10% seronegative human serum was constructed using Recombinant Viral HCMV IL-10 (R&D #117-VL-025) with 2 fold dilution, and PBS with 10% seronegative human serum as blank for an 8th point. Blood samples were tested at 10%: 16 μl plasma+144 μl PBS.

Following incubation, plates were washed 3× with wash buffer. Detection antibody (Viral HCMV IL-10 Biotinylated Affinity Purified Pab, Goat IgG (R&D #BAF117)) was then added at 0.2 μg/ml in PBS, seal with adhesive cover, and incubated for 2 hours at RT on a shaker followed by washing 3× with wash buffer. The samples were then incubated with 1:200 Streptavidin-HRP (R&D #DY998, or as indicated by vendor instructions) in PBS for 20 minutes at RT on a shaker (cover with foil to avoid exposure to light) followed by washing 3× with wash buffer. Substrate Solution (R&D #DY999) was then incubated for 20 minutes max at RT (avoid direct light exposure) followed by addition of stop solution (1M H₂SO₄). Plates were read at 450 nm within 30 minutes.

Extracting Genomic DNA (gDNA) from Whole Blood:

Genomic DNA was isolated from whole blood for PCR using the Promega ReliaPrep Blood gDNA Miniprep System (Part #TM330) based on the manufacturer's protocol. Two separate preps were typically performed for each blood sample. The blood sample was thoroughly mixed for 10 minutes (min) in a rotisserie shaker at room temperature (RT). About 20 μl of Proteinase K (PK) was dispensed into a 1.5 ml centrifuge tube followed by addition of 200 μl of whole blood which was briefly mixed. 200 μl of Cell Lysis Buffer (CLD) was added to the tube and vortexed for 10 seconds. The tube was then incubated at 56° C. for 10 min. The contents of the tube were then added to the ReliaPrep Binding Columnt and placed in a microcentrifuge for 1 min at max speed. The binding column was then placed into a fresh collection tube and 500 μl of Column Wash Solution (CWD) was added to the column 3×, followed by centrifugation for 3 min at maximum speed. 50 μl of nuclease-free water was added to the column and centrifuged for 1 min at max speed to elute gDNA.

Nested PCR on Genomic DNA (gDNA) Using IE1 Primers:

HCMV viral load was determined by detecting IE1 in gDNA of donor blood. This protocol was based on the TaKaRa Ex Taq recommended reaction mixture (Cat# RR001A).

Primers utilized for PCR experiments are shown in Table 1.

TABLE 1
Primer sequences

| Gene | Fwd/Rev | | Amplicon | |
|---|---|---|---|---|
| Target | Primer | Sequence (5′-3′) | length (bp) | Reference |
| beta- | Fwd | GCACCATCCTCCTCTTCC | 256 | IDT ACC# |
| actin | Rev | GGCCTCTGATAACCAAGCC | | ef036500.1 |
| | | | | Assay Set 3 |
| IE1 F1 | Fwd 1 | GGTCACTAGTGACGCTTGTATGATGACCATGTACCGA | 373 | Taylor- |
| IE1 R1 | Rev 1 | GATAGTCGCGGGTACAGGGGACTCT | | Wiedeman, et. |
| IE1 F2 | Fwd 2 | AAGTGAGTTCTGTCGGGTGCT | 293 | al., 1991 |
| IE1 R2 | Rev 2 | GTGACACCAGAGAATCAGAGGA | | |

Two rounds of PCR were performed. The first round of PCR used the outer IE1 primers (IE1 F1 and IE1 R1) and beta actin positive control primers for each sample. The second round of PCR used the first round of PCR as DNA template and the inner IE1 primers (IE1 F2 and IE1 R2). For the first round of PCR, the reaction mixture included 0.50 μL of TaKaRa ExTaq (5 units/ul), 5 μl 10×Ex Taq Buffer, 4 μl dNTP Mixture (2.5 mM each), <500 ng gDNA, 1 μM of each primer, and molecular biology grade water up to 50 μl. PCR reaction tubes were placed in a BioRad MyCycler Thermal Cycler and samples were run under the following PCR conditions: 95° C. 5 min; 35 cycles of 94° C. 30 s, 58° C. 30 s, 72° C. 60 s; 72° C. 5 min. PCR Round 1 products may be visualized on 1% agarose gel electrophoresis. For the second round of PCR, the reaction mixture included 0.25 μL of TaKaRa ExTaq (5 units/ul), 2.5 μl 10×Ex Taq Buffer, 2 μl dNTP Mixture (2.5 mM each), <500 ng gDNA, 1 μM of each primer, and molecular biology grade water up to 25 μl. PCR reaction tubes were placed in a BioRad MyCycler Thermal Cycler and samples were run under the following PCR conditions: 95° C. 5 min; 30 cycles of 94° C. 30 s, 58° C. 30 s, 72° C. 50 s; 72° C. 5 min. PCR products were run on a 1% agarose gel and visualized using ethidium bromide, a BioRad ChemiDic MP Imaging System, and Image Lab 4.0 Software.

Results:

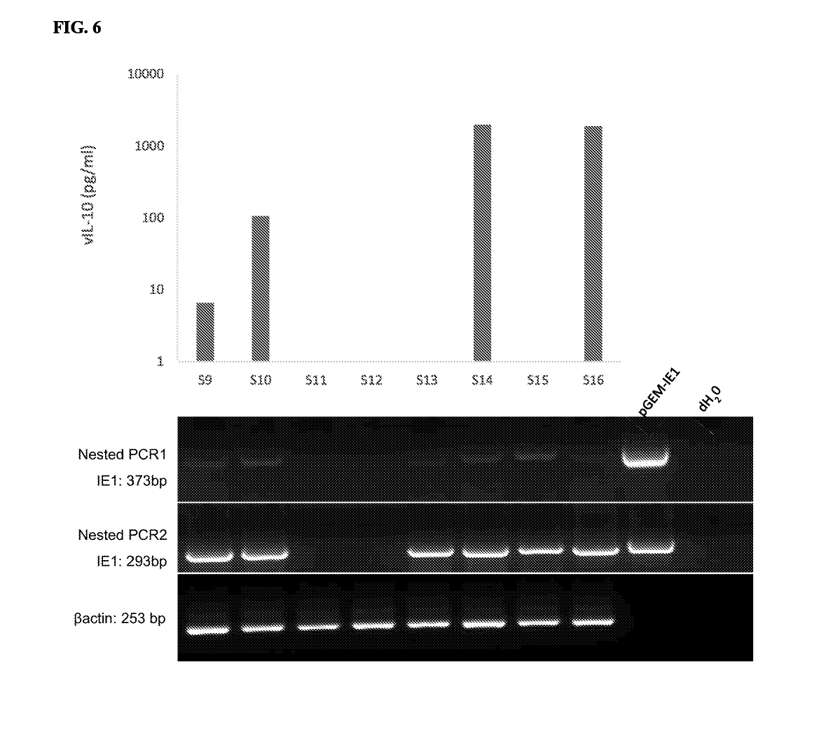

Four blood samples were HCMV seronegative (S9-S12) and four samples were HCMV seropositive (S13-16). vIL-10 levels as determined by ELISA are shown in the bar graph in FIG. 6 (top). vIL-10 was detected in two seronegative specimens. The seronegative donors with vIL-10 lacked any measurable IgG or IgM response to HCMV (Trinity Bioscience ELISA), but viral DNA could be detected by PCR. A nested PCR procedure was used to detect exon 4 of HCMV IE1 on genomic DNA isolated from the whole blood sample, and the top panel of FIG. 6 (bottom) represents first round of PCR. The second round of PCR shown in the middle panel (bottom) of FIG. 6 shows that two seronegative donors have viral DNA present in their blood, which correlates with detection of vIL-10 protein. β-actin as a control (FIG. 6 (bottom, lower panel)). These results suggest that vIL-10 is produced at measurable levels in healthy adults and that seronegative donors may still harbor HCMV.

### Example 6: cmvIL-10 Enhances CXCL12/CXCR4 Calcium Mobilization and Migration

This example demonstrates that cmvIL-10 amplifies CXCR4-mediated calcium signaling in a dose-dependent manner and that cmvIL-10 significantly enhances chemotaxis toward CXCL12.

Materials and Methods:

Cells and Viruses:

Human embryonic kidney (HEK293, American Type Culture Collection, Manassas, Va.) and neonatal foreskin fibroblasts (NuFFs, MTI-GlobalStem, Gaithersburg, Md.) were cultivated in Eagles Minimal Essential Media (MEM) with 10% fetal bovine serum (FBS, Atlanta Biologicals, Norcross, Ga.). All cells were maintained at 37° C. in a humidified incubator with an atmosphere of 5% CO₂.

Calcium Flux Assay:

Calcium flux assays were performed as described (Arnolds K L, et al. Virology 439:122-131.). Briefly, cells were resuspended in 2 mls calcium assay buffer (RPMI with 25 mM of HEPES) at a density of 5×10⁵ cells/ml per sample. Each sample was labeled with 2 μl of Fluo-4AM (Invitrogen, Grand Island, N.Y.), wrapped in foil, at 37° C. for 30 minutes with agitation at 10-minute intervals. Following incubation, each sample was pelleted, resuspended in calcium assay buffer, and transferred into an eppendorf tube for flow cytometry using the BD C6 Accuri. Fluorescence intensity of each sample was measured for 20 seconds to establish baseline, then stimulus (CXCL12, in the presence or absence of cmvIL-10, as indicated) was added via micropipetting directly into the sample while fluorescence intensity was measured. Kinetic fluorescence intensity analysis was performed with FlowJo software v9.4.9 (FlowJo, Ashland, Oreg.). Purified recombinant cmvIL-10 was purchased from R&D Systems (Minneapolis, Minn.) and purified recombinant CXCL12 was purchased from Peprotech (Rocky Hill, N.J.). For each calcium flux assay, ionomycin (Sigma) was a positive control for Fluo-4AM loading and PBS served as a negative control for the addition of stimulus.

Cell Migration Assay:

Transwell migration assays were performed by suspending cells at a density of 5×10⁴ cells/ml in migration media (MEM or RPMI 1650 media plus 0.5% FBS). A total of 75 μl of cells was placed onto the upper chamber of a 96-well transwell system with 5.0 μm pores (Corning-Costar, Corning, N.Y.). The lower chamber contained 235 μl of migration media with CXCL12 plus or minus cmvIL-10 as indicated. Plates were incubated for four hours at 37° C., and then cells that traversed through the filter into the lower chamber were quantified via the addition of CellTitre-Glo reagent (Promega, Madison, Wis.) according to manufacturer's instructions.

Results

cmvIL-10 Enhances Calcium Mobilization by CXCR4 in Response to CXCL12:

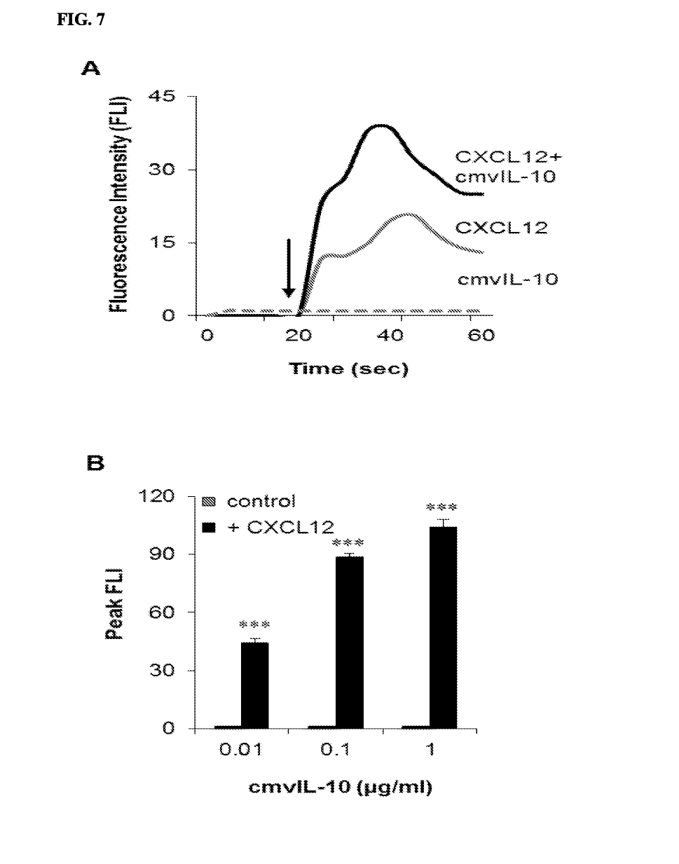

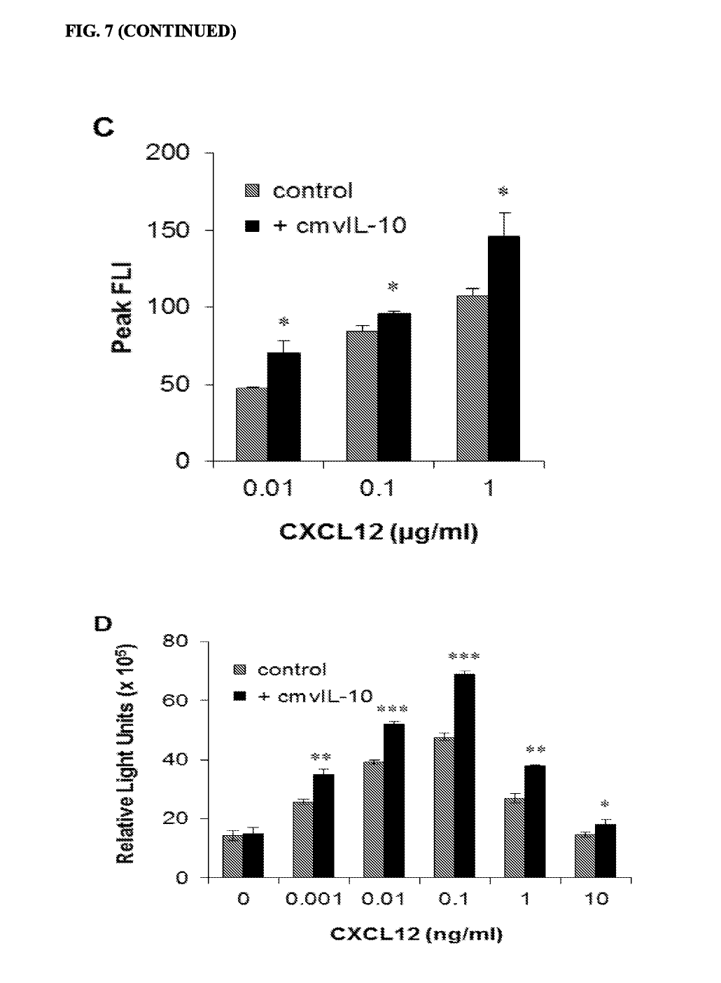

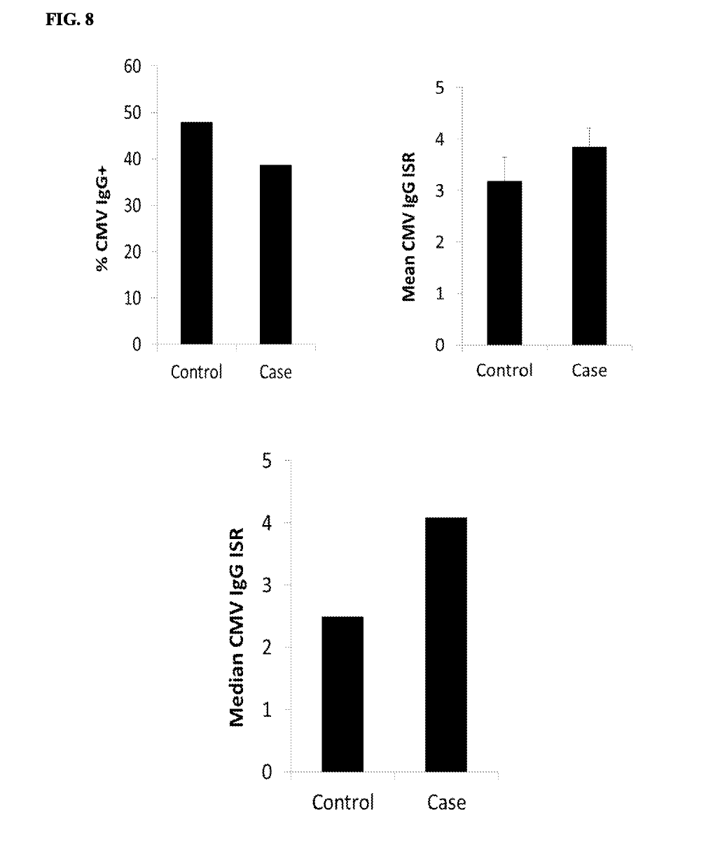

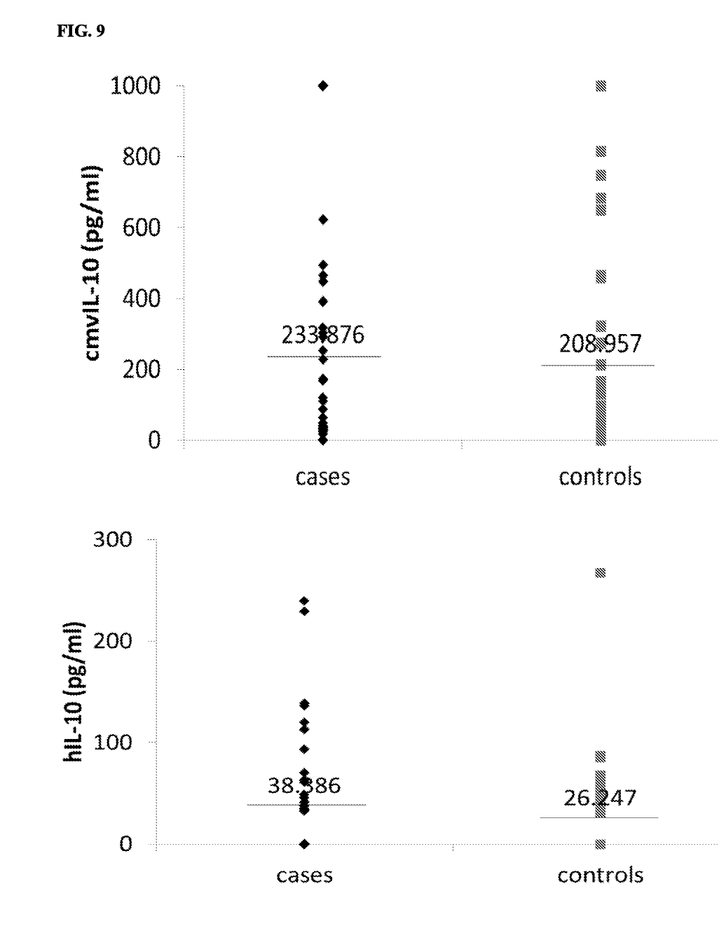

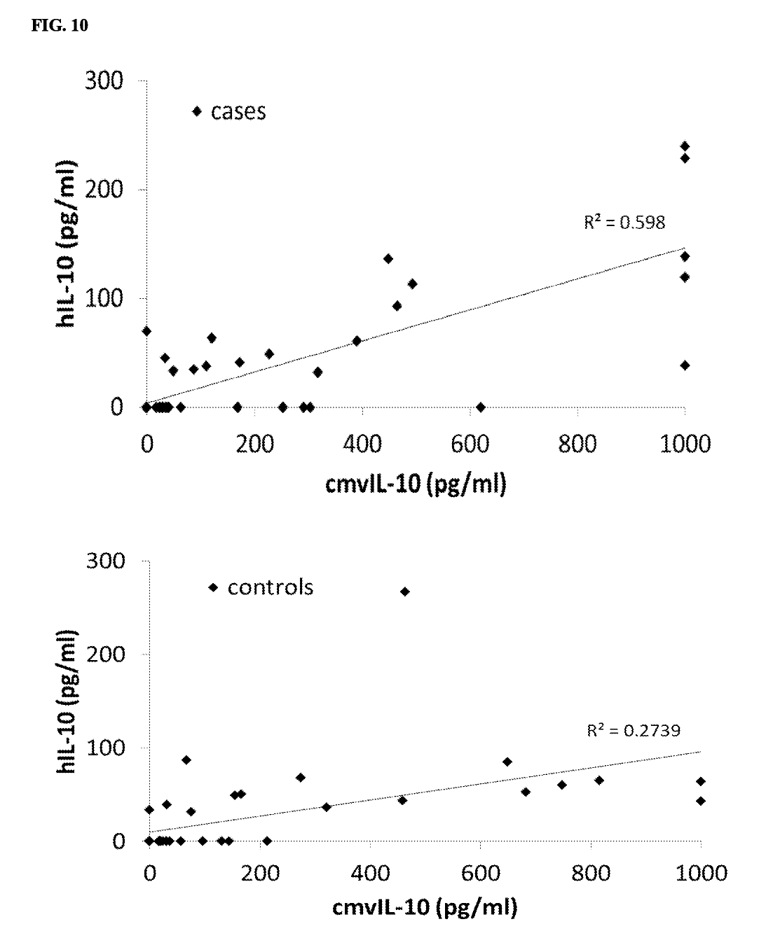

To investigate whether cmvIL-10 could augment the signaling activity of CXCR4, HEK293 cells were loaded with a calcium indicator dye, and then stimulated with CXCL12. Chemokine binding induced the release of sequestered calcium ions into the cytosol, causing a rapid but transient increase in fluorescence, as shown in FIG. 7A. When cells were treated with CXCL12 in the presence of cmvIL-10, there was increased calcium mobilization, and the peak fluorescence intensity was significantly higher. The viral cytokine alone did not induce calcium mobilization (FIG. 7A, B); however, when the CXCL12 concentration was fixed at 0.1 μg/ml, the magnitude of the calcium response increased as the dose of cmvIL-10 increased (FIG. 7B). In addition, cmvIL-10 was effective at increasing calcium flux in response to a range of doses of CXCL12 (FIG. 7C). These results demonstrate that the UL111A gene product, cmvIL-10, amplifies CXCR4-mediated calcium signaling in a dose-dependent manner.

cmvIL-10 Augments Chemotaxis Towards CXCL12:

In order to determine whether cmvIL-10 could enhance downstream signaling outcomes of CXCR4, such as chemotaxis, transwell migration assays were performed. HEK293 cells were placed in the upper chamber, separated from the lower chamber containing CXCL12 by a 5.0 μm pore size filter. After four hours, cells that traversed the filter were collected and quantified. The classic bell-shaped curve for chemotaxis was observed, with maximal migration toward 0.1 ng/ml CXCL12 and less cell movement at higher and lower doses (FIG. 7D). While the basal movement of cells was comparable in the presence or absence of cmvIL-10, migration toward CXCL12 was significantly increased when cmvIL-10 was present. Although the dose of CXCL12 eliciting maximal chemotaxis remained unchanged, the number of migrating cells increased when cmvIL-10 was included in the lower chamber. These experiments were conducted with HEK293 cells, which endogenously express both CXCR4 and IL-10R, and similar results were also observed with THP-1 and U937 monocytic cell lines. Taken together, these findings indicate that cmvIL-10 significantly enhances chemotaxis toward CXCL12.

### Example 7: Women with Breast Cancer have Higher Anti-CMV Antibody Titers

This Example demonstrates that women with breast cancer have higher anti-CMV antibody titers.

Materials and Methods

Cell and Viruses:

CmvIL-10 is secreted from HCMV AD169-infected human newborn foreskin fibroblast (NuFF-1) cells into the cell culture supernatant. Samples of supernatant were collected to measure cmvIL-10 during in vitro infection.

ELISA:

A 96-well microplate was coated overnight at 4° C. with goat polyclonal antibody (Ab) diluted in PBS. Biotinylated goat polyclonal Ab was used to detect cmvIL-10 present in sample, followed by streptavidin-HRP and substrate solution (R&D Systems). The plate was read at 450 nm and concentrations interpolated from a standard curve.

Collection of Specimens.

Female volunteers from USF and the Avon Army of Women with or without a diagnosis of breast cancer within the past 5 years gave their informed consent to participate in the study. Participants completed a medical history questionnaire and approximately 12 mls of blood were collected from each volunteer via standard venipuncture. The blood was processed to collect plasma. This study was approved by the Institutional Board for the Protection of Human Subjects (IRBPHS Protocol 233). Results from plasma testing and medical history questionnaires were analyzed using R and R studio software.

Results

Higher titers of anti-CMV antibodies were found in seropositive cases compared to controls. This could be due to more recent infection or virus reactivation in women with breast cancer. Higher mean levels of cmvIL-10 were detected in plasma from cases compared to controls and the correlation with hIL-10 levels was also stronger in cases. This is consistent with reported findings that cmvIL-10 can induce production of hIL-10 by immune cells (Chang et al. 2004. J. Virology, 78: 8720-31; Rosenberg-Hasson et al. 2014. Immunologic research, 58: 224-33; Avdic et al. 2016. Journal of Virology 90:3819-3827), which may have significance in breast cancer patients. Preliminary analysis of survey responses indicates that age, family history of uterine cancer, and abnormal pap smear are associated with breast cancer.

### Example 8: Human Cytomegalovirus Interleukin-10 Regulates Metastasis-Related Genes and Enhances Invasion of MDA-MB-231 Breast Cancer Cells

This example demonstrates the effects of cmvIL-10 on tumor cell invasion. Using transcriptional profiling, it was shown that cmvIL-10 altered expression of several genes implicated in metastasis.

Materials and Methods

Cell, Viruses, and Reagents:

MDA-MB-231 human breast adenocarcinoma cells (American Type Culture Collection, Manassas, Va.) were cultured in L-15 Leibovitz's Medium (Mediatech, Manassas, Va.) supplemented with 10% fetal bovine serum (FBS) (Atlanta Biologicals, Flowery Branch, Ga.) at 37° C. with atmospheric CO₂. The human foreskin fibroblast (HFF) cell line (ATCC) was cultured in Dulbecco's Modified Eagle Medium (DMEM) with 10% FBS at 37° C. in a humidified chamber with 5% CO₂. Human cytomegalovirus strain AD169 (ATCC) was used to infect confluent monolayers of HFFs at the indicated multiplicities of infection. Purified recombinant cmvIL-10, human IL-10, and epidermal growth factor (EGF) were purchased from R&D Systems (Minneapolis, Minn.). IL-10R neutralizing antibody and S31-201 Stat3 inhibitor were from Santa Cruz Biotechnology (Santa Cruz, Calif.).

Migration and Invasion Assays:

Transwell migration was monitored using 96-well BD Fluoroblock plates with 8 μm filters (Corning, Inc., Corning, N.Y.). Cells were harvested and suspended at density of 2.0×10⁶ cells/ml in migration media (L-15+1% FBS), and a volume of 75 μl cell suspension was placed on top of the filter inserts. Where indicated, IL-10R neutralizing antibody was added at a concentration of 30 μg/ml. The bottom wells were loaded with the indicated concentrations of EGF in the presence of conditioned medium from mock or HCMV-infected fibroblasts (96 hours post infection) in a total volume of 235 μl. After 5 hrs at 37° C., cells that traversed the filter and entered the lower chamber were quantified by the addition of Cell Titer Glo (Promega, Madison, Wis.) using a Turner Veritas luminometer. For invasion, 96-well matrigel-coated BD Fluoroblock transwell invasion plates (Corning) were used. Invasion plates were re-hydrated with warm media at 37° C. for 3 hrs and then 75 μl cell suspension loaded onto the hydrated filters as described above. Where indicated, 10 μM Stat3 inhibitor was included with cells in the top chamber; cmvIL-10, hIL-10 or conditioned medium was present in both chambers. The bottom plates received the indicated EGF concentrations, and then transwell system was incubated for 22 hrs at 37° C. with atmospheric CO₂. At harvest, cells that had degraded the matrigel and entered the lower chamber were quantified by the addition of Cell Titer Glo as above.

Quantitative PCR Arrays:

RNA was harvested from 10×10⁶ MDA-MB-231 cells that were mock treated or treated with 100 ng/mL cmvIL-10 or hIL-10 for 5 hrs using the RNeasy Midi Kit and RNAse-Free DNase set (Qiagen, Valencia, Calif.). From the isolated RNA, cDNA was prepared using the RT² First Strand Kit (SA Biosciences, Frederick, Md.) and subsequently loaded into a 96-well breast cancer metastasis profiler PCR array (PAHS-028ZD) with system RT² SYBR Green Mastermix (SA Biosciences). The plates were run using the CFX96 Real-Time system cycler (BioRad, Hercules, Calif.) with the following amplification program: 95° C. for 10 min, 95° C. for 15 min with a slow ramp rate for 1.0 c/sec and 60° C. for 1 min. The data from three biological replicates for each treatment was analyzed by the ΔΔCT method according to manufacturer's instructions using the RT² profiler PCR array data analysis program located on the SABiosciences web portal and is reported as fold change relative to control.

Enzyme-Linked Immunosorbent Assay (ELISA):

DuoSet ELISA kits (R&D Systems) were used to quantify uPAR, PAI-1, and MMP-3. For uPAR and PAI-1 measurement, MDA cells were seeded in triplicate in 96-well plate at 5.0×10⁴ cell/mL density with complete L-15 media and treated with 10 ng/mL of either cmvIL-10 or hIL-10 for the indicated times and supernatants were collected daily. The ELISA was carried out on supernatants according to manufacturer's instructions using and following the addition of substrate and stop solution, absorbance of the plate was measured at 450 nm using a Dynex Opsys MR microplate reader. Sample concentrations were interpolated from a standard curve using linear regression analysis. For cell-associated MMP-3, MDA cells were seeded in 96-well plates and treated with cmvIL-10 as above. Cells were treated with cell lysis buffer (150 mM NaCl, 20 mM HEPES, 0.5% Triton-X-100, 1.0 mM NaOV₄, 1.0 mM EDTA, 0.1% NaN₃) supplemented with 1× protease inhibitors (Calbiochem, EMD Chemicals, San Diego Calif.) and were collected daily for the indicated time points. The lysates were evaluated for MMP-3 according to the manufacturer's instructions (R&D Systems).

Western Blotting and Zymography:

Confluent T-75 flasks of MDA-MB-231 cells were treated with 10 ng/mL cmvIL-10 (R&D systems) for the indicated times, then scraped and harvested into cell lysis buffer (150 mM NaCl, 20 mM HEPES, 0.5% Triton-X-100, 1.0 mM NaOV₄, 1.0 mM EDTA, 0.1% NaN₃) containing 1× protease inhibitors (Calbiochem). Cell lysates were clarified via centrifugation, heated at 70° C. for 10 min in reducing buffer, and the proteins separated on a 4-12% Tris-Base SDS-PAGE gel (Life Technologies, Grand Island, N.Y.). After transfer to nitrocellulose, the membrane was incubated in blocking solution (5% milk+TBS) for 1 hr at room and then probed with primary antibody: 1:1000 dilution for MMP-3 or MTSS-1 antibodies (Santa Cruz), or MAPK antiserum (Cell Signaling Tech, Danvers, Mass.), in blocking solution overnight, oscillating on a platform rocker at 4.0° C. After three washes, the membranes were incubated with a 1:2000 dilution of appropriate AP-conjugated secondary antibody on a platform rocker at room temperature for 1 hr. Protein bands were detected using western blue stabilized AP substrate (Promega, Madison, Wis.). For zymography, cell lysates were denatured in SDS buffer under non-reducing conditions without heat, and run on a 4-16% Zymogram gel using Tris-Glycine SDS running buffer according to manufacturer's instructions. After electrophoresis, the enzyme was renatured by incubating the gel in Zymogram Renaturing Buffer containing a non-ionic detergent, then equilibrated in Zymogram Developing Buffer (to add divalent metal cations required for enzymatic activity), and then stained and destained to reveal digested (clear) areas corresponding to active enzyme.

Immunofluorescence Microscopy:

MDA-MB-231 cells were seeded onto FBS-coated glass coverslips at a density of 2.0×10⁵ cells/well and cultured for 48 hrs at 37° C. Cells were treated with 100 ng/mL of purified recombinant cmvIL-10 for 96 hrs, then fixed with 2% paraformaldehyde in DPBS for 20 min, washed, permeabilized with 0.2% Triton-X-100 in PBS for 15 min. The cells were washed and blocked with 10% FBS for 1 hr at 37° C., then incubated with anti-MTSS-1 antibody at a 1:100 dilution for 1 hr at 37° C. followed by the addition of goat anti-mouse TRITC secondary antibody at a 1:150 dilution for 30 min (Life Technologies) and Alexa Fluor 488 phalloidin (Molecular Probes, Eugene, Oreg.). Coverslips were washed and excess fluid was removed before inverting the coverslip onto a glass slide containing 20 μL of DAPI-containing Prolong Gold mounting medium (Life Technologies, Grand Island, N.Y.). Images were taken on a Zeiss AX10 Imager.A1 microscope (Carl Zeiss Inc., Oberkochen, Germany) using AxioVision 4.7.2 imaging software.

Statistical Analysis:

Statistical analyses were performed using the paired, two-tailed Student's t-test.

Results

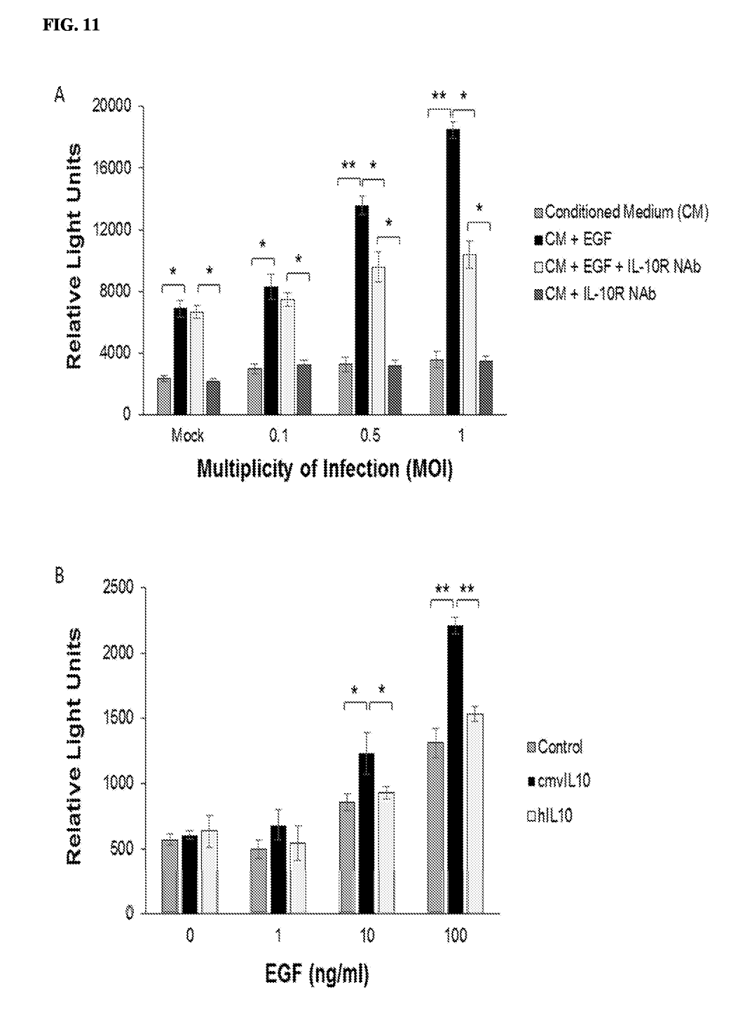

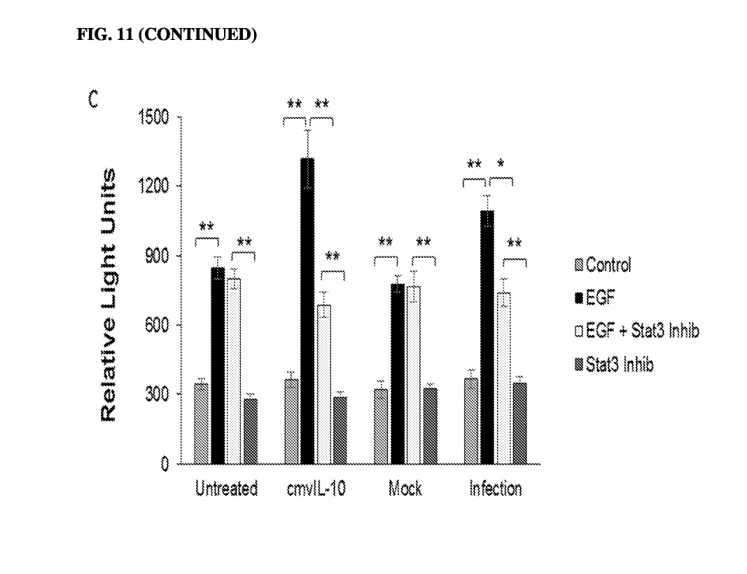

The tumor microenvironment is a complex milieu that includes not only malignant cells, but immune cells, fibroblasts, signaling molecules, the extracellular matrix (ECM), and blood vessels. We have previously found that cmvIL-10 enhances migration of MDA-MB-231 breast cancer cells in vitro toward epidermal growth factor (EGF) (Valle Oseguera Calif., Spencer J V. 2014. PLoS One 9:e88708.). Since those experiments utilized purified recombinant cmvIL-10, we wanted to more faithfully replicate conditions under which cmvIL-10 might be found in the tumor microenvironment. Here we examined the ability of cmvIL-10 secreted from virus-infected cells to stimulate movement of MDA cells. Monolayer cultures of human foreskin fibroblasts were mock-infected or infected with HCMV strain AD169 at a range of multiplicities of infection (MOI). After 96 hours, supernatants were harvested and placed in the lower chamber of a transwell migration plate in the presence or absence of EGF. MDA cells were placed in the upper chamber, separated from the EGF and conditioned medium by a porous filter. After five hours, cells that traversed the filter were quantified. MDA cells did not exhibit any significant movement toward conditioned medium from mock or infected cells, which is consistent with our previous finding that cmvIL-10 is not a chemoattractant for tumor cells (Valle Oseguera Calif., Spencer JV. 2014. PLoS One 9:e88708.). However, when conditioned medium from mock infected cells was supplemented with EGF, cell migration was observed (FIG. 11A). When EGF was added to conditioned medium from HCMV-infected cells, the amount of cell migration increased, suggesting that substances released from virus-infected cells amplified chemotaxis to EGF. Moreover, the enhanced MDA cell movement was greater when EGF was provided in supernatants from higher MOI infections, and thus greater concentrations of cmvIL-10, indicating a dose-dependent effect. To confirm that cmvIL-10 was the virally produced substance mediating this increase in cell movement, MDA cells were pre-incubated for 30 min with a neutralizing antibody (NAb) directed at the cellular IL-10R. The NAb was also included in the top chamber with MDA cells during the five hour incubation, and resulting migration was reduced to levels seen when only EGF was present in medium from mock infected cells. These results demonstrate that cmvIL-10 secreted from virally infected cells has the ability to interact with the cellular IL-10R on tumor cells to enhance directed movement.

To further recapitulate the tumor microenvironment, we examined whether cmvIL-10 could also promote invasion through matrigel, a gelatinous protein mixture derived from mouse sarcoma cells widely used to simulate the ECM in vitro (Hall D M, Brooks S A. 2001. Metastasis Research Protocols: Volume II: Analysis of Cell Behavior In Vitro and In Vivo doi:10.1385/1-59259-137-x:061. Humana Press, Totowa, N.J.). MDA cells were place atop a matrigel-coated transwell system with EGF placed in the lower chambers. Purified recombinant cmvIL-10 or hIL-10 was added to both chambers. After incubation for 22 hours, invasion was assessed by counting cells in the lower chamber, which should contain only the cells that were able to degrade the matrigel coating to access the porous filter. As shown in FIG. 11B, cmvIL-10 was found to be a strong enhancer of cell invasion. Surprisingly, cmvIL-10 was able to increase invasion of MDA breast cancer cells to a significantly greater extent than hIL-10, suggesting that the viral cytokine may trigger signaling events that are distinct from the cellular cytokine. Since activation of the transcription factor Stat3 by cmvIL-10 is well-documented (Kotenko S V, et al. 2000. Proc Natl Acad Sci USA 97:1695-1700; Gruber S G, et al. 2008. Eur J Immunol 38:3365-3375; Raftery M J, et al. 2004. J Immunol 173:3383-3391; Spencer J V. 2007. J Virol 81:2083-2086; Lin Y L, et al. 2008. Virus Res 131:213-223; Avdic S, et al. 2008. J Virol doi:10.1128/JVI.03066-15; Jenkins C, et al. 2008. J Virol 82:3736-3750), we next examined the need for Stat3 in cmvIL-10-enhanced invasion. Treatment with a Stat3 inhibitor reduced the cmvIL-10-induced increase in invasion through matrigel toward EGF seen when either recombinant purified protein or cytokine produced during virus infection were present (FIG. 11C). Taken together, these results demonstrate that cmvIL-10 produced during virus infection stimulates enhanced migration and invasion of breast cancer cells.

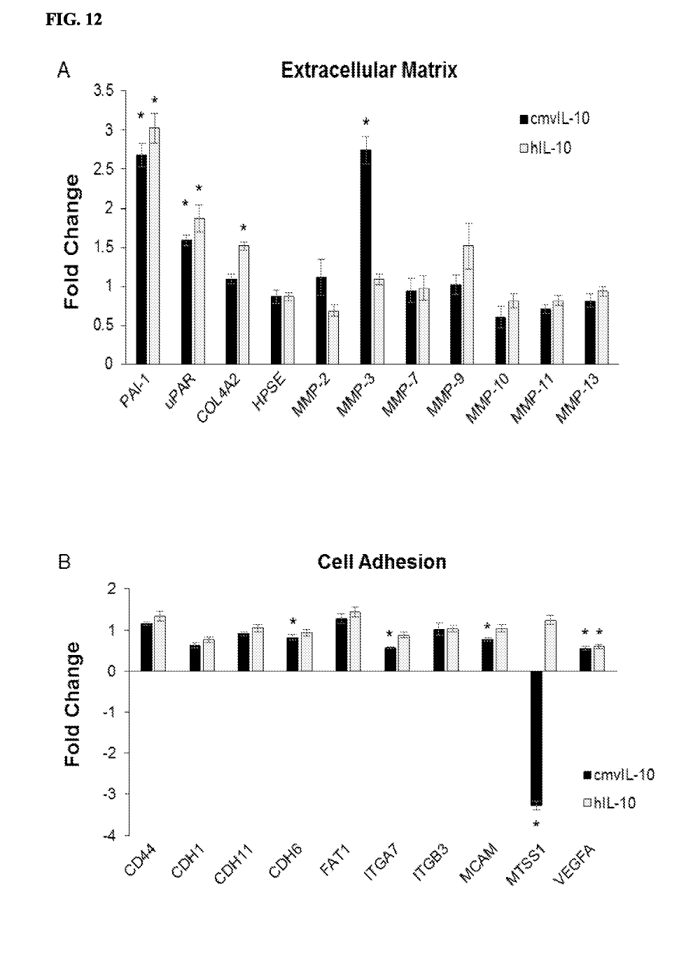

Given the impact of cmvIL-10 on MDA cell invasion, we wanted to investigate whether the viral cytokine brought about changes in the expression of genes associated with tumor metastasis. Transcriptional profiling was performed using a tumor metastasis array designed to analyze 84 genes known to be involved in breast cancer metastasis. MDA cells were mock-treated or incubated with either cmvIL-10 or hIL-10 for 5 hours, then RNA was extracted, cDNA synthesized, and qPCR performed. Table 2 contains a complete list of genes analyzed with fold changes for cmvIL-10 or hIL-10 treated cells compared to mock treated control cells indicated. Select genes encoding proteins associated with either the ECM (FIG. 12A) or cell adhesion (FIG. 12B) are shown graphically. Overall, plasminogen activator inhibitor (PAI-1) was the most highly upregulated gene for both cytokines, with expression increased by 2.68-fold by cmvIL-10 and 3.12-fold by hIL-10. Interestingly, increased expression of urokinase plasminogen receptor (uPAR) was also common to both cmvIL-10 and hIL-10 (1.59 and 1.87 fold increases, respectively). Matrix metalloproteinase-3 (MMP3) was specifically upregulated by cmvIL-10 only (2.75 fold increase), while collagen type 4 (COL4A) expression was increased by hIL-10 only (1.51 fold increase). Changes in cell adhesion genes were more modest, with only one gene, metastasis suppressor 1 (MTSS1) exhibiting a statistically significant change of more than 2-fold, and this was observed for cmvIL-10 treatment only (0.305 fold change, or −3.28). Slight decreases in integrin alpha 7 (ITGA7, 0.561 or −1.78 fold change), melanoma cell adhesion factor (MCAM, 0.768 or −1.32 fold change), and cadherin 6 (CDH6, 0.811 or −1.23 fold change) were also found with cmvIL-10 treatment. Both cmvIL-10 and hIL-10 induced a slight decrease in expression of vascular endothelial growth factor (VEGFA, 0.554, or −1.80 fold for cmvIL-10; 0.5987 or 1.67 fold change for hIL-10). Chemokine receptor CXCR2 expression was also strongly decreased by cmvIL-10 and hIL-10, but those changes were not statistically significant. Overall, these transcriptional profiling results indicate that cmvIL-10, as well as human IL-10, can affect expression of genes that are likely to promote metastatic spread of tumor cells.

TABLE 2
Transcriptional Profiling of MDA Cells Exposed to cmvIL-10 or hIL-10

| | | | | Fold Change vs. Mock | |
|---|---|---|---|---|---|
| Unigene | Refseq | Symbol | Description | cmvIL-10 | hIL-10 |
| Hs.158932 | NM_000038 | APC | Adenomatous polyposis coli | 0.9681 | 1.0052 |
| Hs.100426 | NM_015399 | BRMS1 | Breast cancer metastasis suppressor 1 | 0.8029 | 1.1154 |
| Hs.251526 | NM_006273 | CCL7 | Chemokine (C-C motif) ligand 7 | 0.8113 | 0.9314 |
| Hs.502328 | NM_000610 | CD44 | CD44 molecule (Indian blood group) | 1.1473 | 1.3402 |
| Hs.527778 | NM_002231 | CD82 | CD82 molecule | 0.6917 | 1.1019 |
| Hs.461086 | NM_004360 | CDH1 | Cadherin 1, type 1, E-cadherin (epithelial) | 0.6288 | 0.7618 |
| Hs.116471 | NM_001797 | CDH11 | Cadherin 11, type 2, OB-cadherin (osteoblast) | 0.9033 | 1.0425 |
| Hs.171054 | NM_004932 | CDH6 | Cadherin 6, type 2, K-cadherin (fetal kidney) | 0.8113 | 0.9314 |
| Hs.512599 | NM_000077 | CDKN2A | Cyclin-dependent kinase inhibitor 2A (melanoma, p16, | 0.8113 | 0.9314 |
| | | | inhibits CDK4) | | |
| Hs.162233 | NM_001273 | CHD4 | Chromodomain helicase DNA binding protein 4 | 0.8970 | 1.1115 |
| Hs.508716 | NM_001846 | COL4A2 | Collagen, type IV, alpha 2 | 1.0929 | 1.5131 |
| Hs.143212 | NM_003650 | CST7 | Cystatin F (leukocystatin) | 0.7149 | 0.8858 |
| Hs.208597 | NM_001328 | CTBP1 | C-terminal binding protein 1 | 0.6245 | 0.9576 |
| Hs.534797 | NM_001903 | CTNNA1 | Catenin (cadherin-associated protein), alpha 1, 102 kDa | 0.8816 | 0.9428 |
| Hs.632466 | NM_000396 | CTSK | Cathepsin K | 0.8043 | 0.8482 |
| Hs.716407 | NM_001912 | CTSL1 | Cathepsin L1 | 0.8212 | 0.8981 |
| Hs.522891 | NM_000609 | CXCL12 | Chemokine (C-X-C motif) ligand 12 | 0.8113 | 0.9314 |
| Hs.846 | NM_001557 | CXCR2 | Chemokine (C-X-C motif) receptor 2 | 0.3804 | 0.5885 |
| Hs.593413 | NM_003467 | CXCR4 | Chemokine (C-X-C motif) receptor 4 | 0.8084 | 0.8751 |
| Hs.22393 | NM_003677 | DENR | Density-regulated protein | 0.9816 | 0.9626 |
| Hs.523329 | NM_004442 | EPHB2 | EPH receptor B2 | 0.6693 | 0.9028 |
| Hs.434059 | NM_001986 | ETV4 | Ets variant 4 | 0.5418 | 1.0175 |
| Hs.374477 | NM_005243 | EWSR1 | Ewing sarcoma breakpoint region 1 | 0.9465 | 1.3104 |
| Hs.481371 | NM_005245 | FAT1 | FAT tumor suppressor homolog 1 (Drosophila) | 1.2752 | 1.4414 |
| Hs.165950 | NM_002011 | FGFR4 | Fibroblast growth factor receptor 4 | 0.5362 | 0.9693 |
| Hs.646917 | NM_002020 | FLT4 | Fms-related tyrosine kinase 4 | 0.8113 | 1.0317 |
| Hs.203717 | NM_002026 | FN1 | Fibronectin 1 | 1.1044 | 1.2100 |
| Hs.333418 | NM_014164 | FXYD5 | FXYD domain containing ion transport regulator 5 | 0.9400 | 1.0479 |
| Hs.82963 | NM_000825 | GNRH1 | Gonadotropin-releasing hormone 1 (luteinizing-releasing | 0.9970 | 1.1647 |
| | | | hormone) | | |
| Hs.396530 | NM_000601 | HGF | Hepatocyte growth factor (hepapoietin A; scatter factor) | 0.8113 | 0.9314 |
| Hs.44227 | NM_006665 | HPSE | Heparanase | 0.8620 | 0.8645 |
| Hs.37003 | NM_005343 | HRAS | V-Ha-ras Harvey rat sarcoma viral oncogene homolog | 1.0075 | 1.0625 |
| Hs.90753 | NM_006410 | HTATIP2 | HIV-1 Tat interactive protein 2, 30 kDa | 0.7439 | 0.9138 |
| Hs.160562 | NM_000618 | IGF1 | Insulin-like growth factor 1 (somatomedin C) | 0.8635 | 0.9794 |
| Hs.83077 | NM_001562 | IL18 | Interleukin 18 (interferon-gamma-inducing factor) | 0.7362 | 0.7526 |
| Hs.126256 | NM_000576 | IL1B | Interleukin 1, beta | 0.6266 | 0.6771 |
| Hs.524484 | NM_002206 | ITGA7 | Integrin, alpha 7 | 0.5609 | 0.8796 |
| Hs.218040 | NM_000212 | ITGB3 | Integrin, beta 3 (platelet glycoprotein IIIa, antigen CD61) | 1.0198 | 1.0317 |
| Hs.95008 | NM_002256 | KISS1 | KiSS-1 metastasis-suppressor | 1.1394 | 1.5856 |
| Hs.208229 | NM_032551 | KISS1R | KISS1 receptor | 0.8113 | 0.9314 |
| Hs.505033 | NM_004985 | KRAS | V-Ki-ras2 Kirsten rat sarcoma viral oncogene homolog | 1.0304 | 0.9931 |
| Hs.599039 | NM_006500 | MCAM | Melanoma cell adhesion molecule | 0.7688 | 1.0353 |
| Hs.484551 | NM_002392 | MDM2 | Mdm2 p53 binding protein homolog (mouse) | 0.9531 | 0.9461 |
| Hs.132966 | NM_000245 | MET | Met proto-oncogene (hepatocyte growth factor receptor) | 0.9833 | 1.1173 |
| Hs.444986 | NM_006838 | METAP2 | Methionyl aminopeptidase 2 | 1.1295 | 1.0736 |
| Hs.651869 | NM_002410 | MGAT5 | Mannosyl (alpha-1,6-)-glycoprotein beta-1,6-N-acetyl- | 1.1795 | 1.3590 |
| | | | glucosaminyltransferase | | |
| Hs.2258 | NM_002425 | MMP10 | Matrix metallopeptidase 10 (stromelysin 2) | 0.5980 | 0.6866 |
| Hs.143751 | NM_005940 | MMP11 | Matrix metallopeptidase 11 (stromelysin 3) | 0.7026 | 1.0867 |
| Hs.2936 | NM_002427 | MMP13 | Matrix metallopeptidase 13 (collagenase 3) | 0.8127 | 0.9777 |
| Hs.513617 | NM_004530 | MMP2 | Matrix metallopeptidase 2 (gelatinase A) | −1.1101 | 1.5157 |
| Hs.375129 | NM_002422 | MMP3 | Matrix metallopeptidase 3 (stromelysin 1, progelatinase) | 2.7478 | 0.8080 |
| Hs.2256 | NM_002423 | MMP7 | Matrix metallopeptidase 7 (matrilysin, uterine) | 0.9416 | 0.8123 |
| Hs.297413 | NM_004994 | MMP9 | Matrix metallopeptidase 9 (gelatinase B) | 1.0233 | 0.9347 |
| Hs.525629 | NM_004689 | MTA1 | Metastasis associated 1 | 0.7388 | 0.9395 |
| Hs.700429 | NM_014751 | MTSS1 | Metastasis suppressor 1 | −3.2818 | 1.2397 |
| Hs.202453 | NM_002467 | MYC | V-myc myelocytomatosis viral oncogene homolog (avian) | 0.8113 | 0.7423 |
| Hs.437922 | NM_005376 | MYCL1 | V-myc myelocytomatosis viral oncogene homolog 1, lung | 0.5260 | 1.0644 |
| | | | carcinoma derived (avian) | | |
| Hs.187898 | NM_000268 | NF2 | Neurofibromin 2 (merlin) | 1.0742 | 1.3496 |
| Hs.118638 | NM_000269 | NME1 | Non-metastatic cells 1, protein (NM23A) expressed in | 1.0358 | 1.1000 |
| Hs.9235 | NM_005009 | NME4 | Non-metastatic cells 4, protein expressed in | 0.8226 | 0.9965 |
| Hs.279522 | NM_006981 | NR4A3 | Nuclear receptor subfamily 4, group A, member 3 | 0.5389 | 0.8151 |
| Hs.466871 | NM_002659 | PLAUR | Plasminogen activator, urokinase receptor | 1.5891 | 1.8628 |
| Hs.409965 | NM_002687 | PNN | Pinin, desmosome associated protein | 1.3088 | 1.2901 |
| Hs.500466 | NM_000314 | PTEN | Phosphatase and tensin homolog | 0.7809 | 0.8645 |
| Hs.408528 | NM_000321 | RB1 | Retinoblastoma 1 | 0.9548 | 0.9298 |
| Hs.494178 | NM_006914 | RORB | RAR-related orphan receptor B | 0.6953 | 0.8827 |
| Hs.449909 | NM_002295 | RPSA | Ribosomal protein SA | 0.9631 | 1.0886 |
| Hs.414795 | NM_000602 | SERPINE1 | Serpin peptidase inhibitor, plasminogen activator inhibitor | 2.6818 | 3.0262 |
| | | | type 1 | | |
| Hs.436687 | NM_003011 | SET | SET nuclear oncogene | 0.9664 | 0.9234 |
| Hs.12253 | NM_005901 | SMAD2 | SMAD family member 2 | 0.6869 | 0.7859 |
| Hs.75862 | NM_005359 | SMAD4 | SMAD family member 4 | 0.8384 | 0.8279 |
| Hs.195659 | NM_005417 | SRC | V-src sarcoma (Schmidt-Ruppin A-2) viral oncogene | 0.5297 | 0.9760 |
| | | | homolog (avian) | | |
| Hs.514451 | NM_001050 | SSTR2 | Somatostatin receptor 2 | 0.5556 | 0.7120 |
| Hs.371720 | NM_003177 | SYK | Spleen tyrosine kinase | 0.8590 | 0.9314 |
| Hs.475018 | NM_005650 | TCF20 | Transcription factor 20 (AR1) | 0.7823 | 0.9676 |
| Hs.645227 | NM_000660 | TGEB1 | Transforming growth factor, beta 1 | 0.7173 | 0.9931 |
| Hs.633514 | NM_003255 | TIMP2 | TIMP metallopeptidase inhibitor 2 | 0.7596 | 0.8601 |
| Hs.644633 | NM_000362 | TIMP3 | TIMP metallopeptidase inhibitor 3 | 0.8155 | 0.7631 |
| Hs.591665 | NM_003256 | TIMP4 | TIMP metallopeptidase inhibitor 4 | 0.7324 | 0.9122 |
| Hs.478275 | NM_003810 | TNFSF10 | Tumor necrosis factor (ligand) superfamily, member 10 | 0.8169 | 0.7955 |
| Hs.654481 | NM_000546 | TP53 | Tumor protein p53 | 0.6180 | 0.8873 |
| Hs.155942 | NM_002420 | TRPM1 | Transient receptor potential cation channel, subfamily M, | 0.8113 | 0.9314 |
| | | | member 1 | | |
| Hs.160411 | NM_000369 | TSHR | Thyroid stimulating hormone receptor | 0.8113 | 0.9314 |
| Hs.73793 | NM_003376 | VEGFA | Vascular endothelial growth factor A | 0.5541 | 0.5987 |
| Hs.520640 | NM_001101 | ACTB | Actin, beta | 1.0986 | 1.3287 |
| Hs.534255 | NM_004048 | B2M | Beta-2-microglobulin | 0.9597 | 0.9897 |
| Hs.592355 | NM_002046 | GAPDH | Glyceraldehyde-3-phosphatedehydrogenase | 0.9335 | 1.2121 |
| Hs.412707 | NM_000194 | HPRT1 | Hypoxanthine phosphoribosyltransferase 1 | 1.2796 | 1.3827 |
| Hs.546285 | NM_001002 | RPLP0 | Ribosomal protein, large, P0 | 1.0376 | 1.0000 |

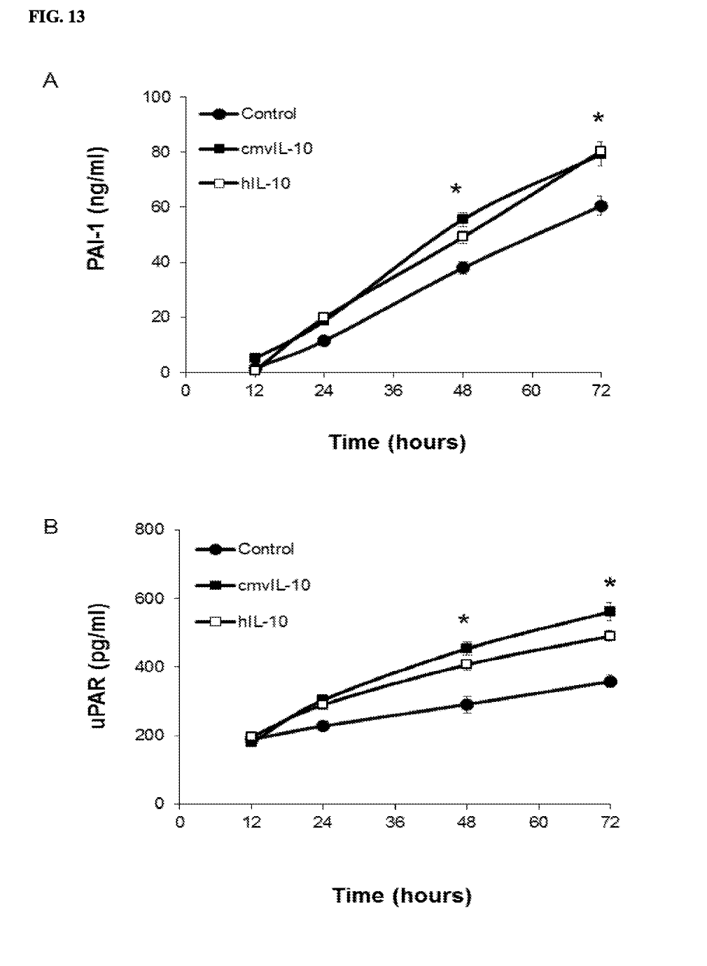

The most significantly upregulated gene by both cmvIL-10 and hIL-10 was PA-1, or plasminogen activator inhibitor 1. PAI-1 is a 43 kDa glycoprotein that inhibits the function of urokinase plasminogen activator (uPA), a serine protease that catalyzes the conversion of inactive plasminogen to plasmin and has been implicated in many aspects of tumor progression (Duffy M J. 2004. Curr Pharm Des 10:39-49.). The activity of uPA system is regulated by the receptor uPAR and two endogenous inhibitors, PAI-1 and PAI-2 (Duffy M J. 2004. Curr Pharm Des 10:39-49.). PAI-1 is constitutively secreted by many cell types and high levels have been found to inhibit cell adhesion and promote migration (Isogai C, et al. 2001. Cancer Res 61:5587-5594; Deng G, et al. 1996. J Cell Biol 134:1563-1571.). In order to confirm that changes in gene expression identified by the qPCR array correlated with protein expression, MDA cells were treated with cmvIL-10 or hIL-10 and PAI-1 levels measured by ELISA. As expected, PAI-1 was produced by untreated cells, however, the amount of protein secreted was significantly increased by cmvIL-10 after 12 hours of exposure (FIG. 13A). After 24 hours, both cmvIL-10 and hIL-10 stimulated a significant increase in PAI-1 production, and this was maintained over 72 hours. In addition, we examined uPAR protein levels and found that they were also elevated upon exposure to cmvIL-10 or hIL-10 (FIG. 13B). These results demonstrate that expression of two elements of the uPA serine protease system, its receptor uPAR and its serpin inhibitor PAI-1, are significantly increased by cmvIL-10 and hIL-10 in human breast cancer cells.

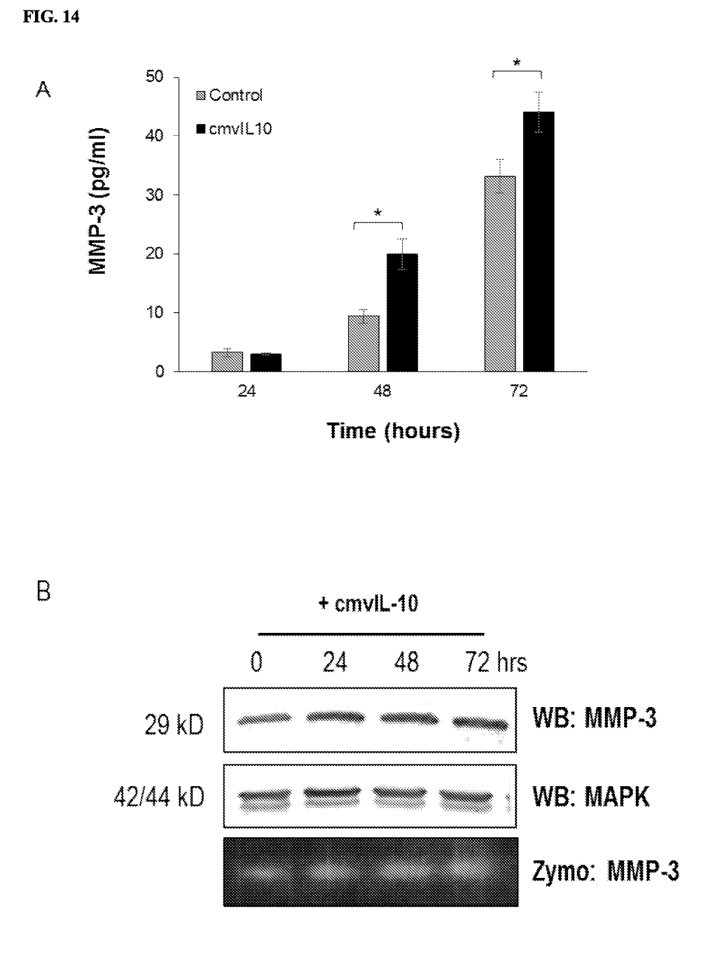

Next we examined MMP-3, a member of the matrix metalloproteinase family that has the ability degrade many components of the extracellular matrix, such as collagen III-V, and IX-XI, as well as laminins, elastins, fibronectin, vitronectins and proteoglycans (Niebroj-Dobosz I, et al. 2010. Eur J Neurol 17:226-231.). Mouse epithelial mammary cells cultured with MMP-3 had decreased expression of cytokeratin markers and increased expression of vimentin, a clear sign of the epithelial-to-mesenchymal transition (EMT), in which epithelial cells morph into a mesenchymal-type cell to eliminate their connection to the basement membrane and initiate migration towards subsequent intravasation into blood vessels (Sternlicht M D, et al. 1999. Cell 98:137-146.). MMP-3 can also activate other MMPs, and high levels of MMP-3 correlate with poor prognosis in breast cancer patients (Duffy M J, et al. 2000. Breast Cancer Res 2:252-257.). MDA cells were treated with cmvIL-10 and then total MMP-3 levels were measured by ELISA. We were unable to detect any MMP-3 in cell supernatants, but cell-associated MMP-3 was detected by analysis of cell lysates. Relatively low levels of MMP-3 were produced by untreated MDA cells, but this amount increased significantly after 48 hours of treatment with cmvIL-10 (FIG. 14A). Since MMPs are generally secreted as inactive pro-enzymes that require cleavage to become activated, we further examined MMP-3 by western blotting and zymography. Consistent with the ELISA results, an increase in total MMP-3 protein was observed over time with exposure to cmvIL-10 (FIG. 14B). The amount of active MMP-3 enzyme was also increased by cmvIL-10 treatment, as evidence by increased digestion of casein in the zymogen gel. Taken together, these results indicate that cmvIL-10 promotes increased expression and activation of MMP-3 by breast cancer cells, which is likely to contribute to increased degradation of the ECM and greater risk of metastasis.

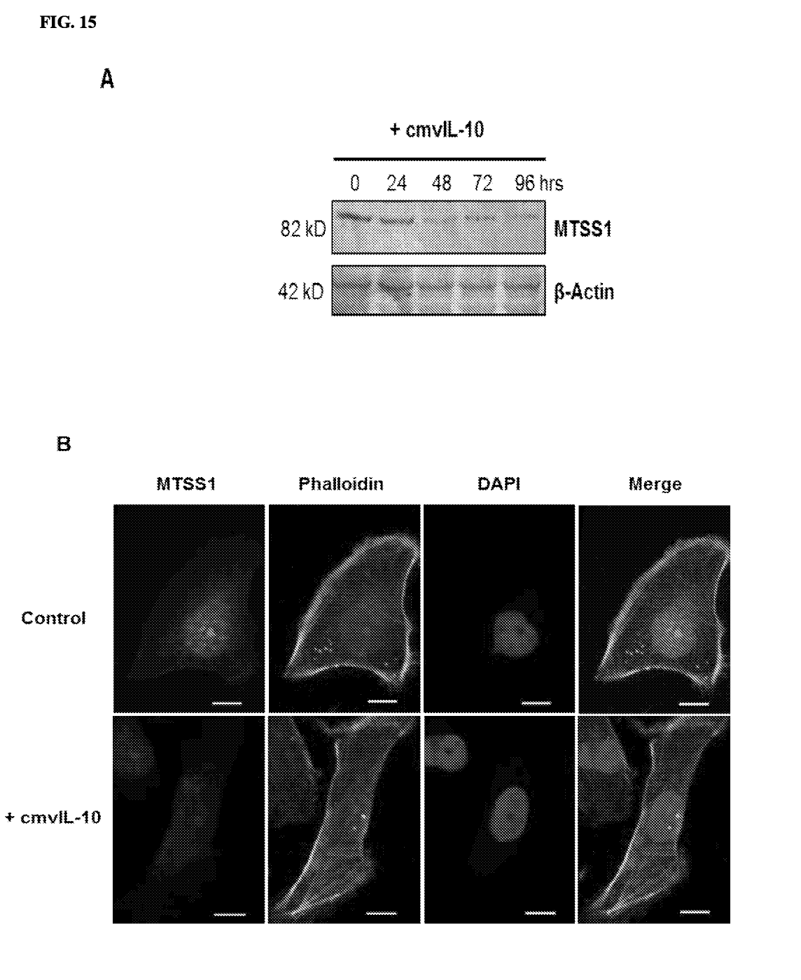

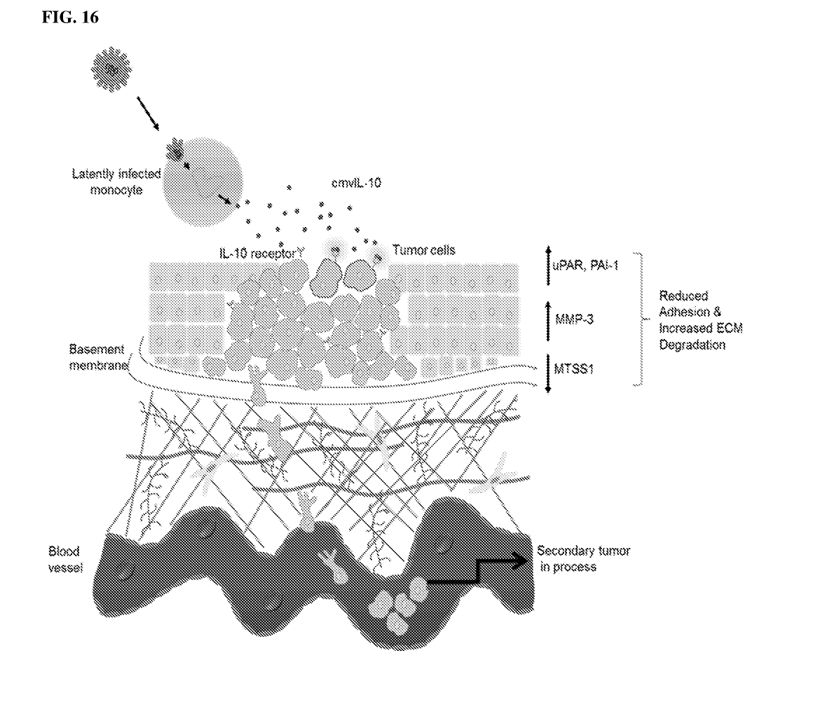

MTSS1 was notable as the gene most strongly downregulated by cmvIL-10 treatment. Also known as missing-in-metastasis (MIM), MTSS1 was originally identified as a tumor suppressor gene whose expression was lost in metastatic bladder and prostate cancers (Lee Y G, et al. 2002. Neoplasia 4:291-294.). The tumor suppressor works as a scaffold to inhibit the dissociation of cell junctions and to increase adherens junction formation, so when MTSS1 is lost recruitment of F-actin to the cytoskeleton is reduced, enabling tumor cells to detach from the basement membrane and from neighboring cells. MTSS1 has been found to be inversely correlated to the aggressive invasive potential in several breast cell lines and with overall survival in breast cancer patients (Parr C, Jiang W G. 2009. Eur J Cancer 45:1673-1683.). To confirm that the reduced gene expression observed on the PCR array correlated with a decrease in MTSS1 protein levels, immunoblotting was performed on lysates from MDA-MB-231 cells treated with 10 ng/mL cmvIL-10. The expected 82 kD band was detected for MTSS1 in untreated cells and was still visible after 24 hours of incubation with cmvIL-10 (FIG. 15A). However, as time progressed, the cmvIL-10-treated samples showed a significant decrease in MTSS1 expression. In contrast, the β-actin bands that serve as a loading control remained constant. We further examined MTSS1 expression via immunofluorescence microscopy and found the protein to be widely distributed throughout the cytoplasm in untreated MDA cells (FIG. 15B), which is consistent with its role in regulating cytoskeletal rearrangement. After exposure to cmvIL-10, dramatic reduction in the amount of MTSS1 protein was observed. This reduction in MTSS1 corresponded to a noticeable change in cellular architecture, as cmvIL-10-treated cells appeared to be thinner and have fewer substrate attachment points. These results demonstrate that treatment with cmvIL-10 reduced the expression of MTSS1 in MDA cells, which could contribute to the increased migration and invasion observed in the presence of cmvIL-10.

We claim:

1. A method for predicting or determining the occurrence of metastasis in an individual affected with breast cancer, the method comprising: detecting the presence of viral interleukin 10 (cmvIL-10) in a biological sample provided by the individual,
wherein the presence of cmvIL-10 indicates that the breast cancer has metastasized and wherein the individual is seronegative for HCMV.

2. A method for determining the risk of metastasis in an individual affected with a breast tumor, the method comprising: detecting the presence of viral interleukin 10 (cmvIL-10) in a biological sample provided by the individual,
wherein the presence of cmvIL-10 indicates that the individual is at increased risk for breast cancer metastasis and wherein the individual is seronegative for HCMV.

3. The method of claim 1 or 2, wherein the sample is selected from the group consisting of a blood sample, a tissue sample, a urine sample, a saliva sample, a semen sample, a tear sample, or a breast milk sample.

4. The method of any one of claims 1-3, wherein cmvIL-10 is detected by detecting a cmvIL-10 nucleic acid in the sample.

5. The method of claim 4, wherein the cmvIL-10 nucleic acid is DNA.

6. The method of claim 5, wherein the cmvIL-10 DNA is detected by PCR or Southern Blotting.

7. The method of claim 4, wherein the cmvIL-10 nucleic acid is RNA.

8. The method of claim 7, wherein the cmvIL-10 RNA is detected by RT-PCR, Northern Blotting, in situ hybridization, microarray, or RNase protection assay.

9. The method of any one of claims 1-3, wherein the cmvIL-10 is detected by detecting a cmvIL-10 protein in the sample.

10. The method of claim 9, wherein the vIL-10 protein is detected by Western Blotting, immunoprecipitation, immunocytochemistry, immunohistochemistry, immunoelectron microscopy, radioimmunoassay, Enzyme-Linked ImmunoSpot (ELISPOT) assay, 2D gel electrophoresis, or enzyme-linked immunosorbent assay (ELISA).

11. The method of claim 10, wherein the cmvIL-10 protein is detected by ELISA.

12. The method of claim 11, wherein the antibody used in the ELISA is a polyclonal antibody.

13. The method of claim 11, wherein the antibody used in the ELISA assay is a monoclonal antibody.

14. The method of any one of claims 1-13, wherein said individual is pre- or post-menopausal.

15. The method of any one of claims 1-13, wherein said individual has been diagnosed as having breast cancer and said method is used to determine if said breast cancer has recurred or advanced.

16. The method of any one of claims 1-13, wherein said individual has not been previously diagnosed as having breast cancer.

17. The method of any one of claims 1-16, wherein cmvIL-10 is LAcmvIL-10.

18. The method of claim 1 or 2, further comprising detecting upregulation of at least one gene selected from the group consisting of plasminogen activator inhibitor 1 (PAI-1), urokinase plasminogen activator (uPA), urokinase plasminogen activator receptor (uPAR), and matrix metalloproteinase-3 (MMP-3), The method of claim 1 or 2, further comprising detecting downregulation of the gene missing-in-metastasis (MTSS).

19. The method of claim 1 or 2, further comprising detecting CXCR4-mediated calcium signaling.

20. The method of claim 1 or 2, further comprising detecting chemotaxis toward CXCL12.

21. A kit for detecting human cytomegalovirus (HCMV) in a sample provided by an individual diagnosed with breast cancer comprising:
a) a probe for detecting the presence of viral interleukin 10 (cmvIL-10) in the sample; and
b) one or more buffers and/or reagents,
wherein the individual is seronegative for HCMV.

22. The kit of claim 21, wherein the probe is selected from the group consisting of a nucleic acid probe or an antibody.

23. The kit of claim 21 or 22, further comprising c) a secondary antibody.

24. The kit of any one of claims 21-23, wherein the antibody or the secondary antibody is conjugated to an enzyme.

25. The kit of any one of claims 21-24, further comprising d) a substrate.

26. The kit of any one of claims 21-25, wherein cmvIL-10 is LAcmvIL-10.

27. A method for detecting human cytomegalovirus (HCMV) in a biological sample provided by an individual, the method comprising: (a) contacting the biological sample comprising viral interleukin 10 (cmvIL-10) with a probe that specifically binds to a cmvIL-10 polypeptide or nucleic acid; and (b) detecting the presence of cmvIL-10 when a complex is formed between the probe and cmvIL-10 polypeptide or nucleic acid, wherein the individual is seronegative for HCMV and wherein the individual has been diagnosed with breast cancer.

28. The method of claim 27, wherein the probe comprises one or more nucleic acids.

29. The method of claim 28, wherein the one or more nucleic acids specifically hybridize to a nucleic acid of SEQ ID NO: 1 or SEQ ID NO: 2.

30. The method of any one of claims 27-29, wherein said one or more nucleic acids are PCR primers and PCR is performed subsequent to the complex forming between the PCR primers and the cmvIL-10 nucleic acid.

31. The method of any one of claims 27-29, wherein the one or more nucleic acids is detectably labeled.

32. The method of claim 27, wherein the probe comprises an antibody or fragment thereof.

33. The method of claim 32, wherein the antibody or fragment thereof is a monoclonal antibody.

34. The method of claim 32, wherein the antibody is a polyclonal antibody.

35. The method of claim 34, wherein the polyclonal antibody is produced using a recombinantly-produced cmvIL-10 polypeptide immunogen comprising A26 to K176 of SEQ ID NO:3.

36. The method of claim 34 or 35, wherein the polyclonal antibody is derived from goat.

37. The method of any one of claims 27-36, further comprising (c) contacting the biological sample with a probe that specifically binds to one or more polypeptides or nucleic acids selected from the group consisting of plasminogen activator inhibitor 1 (PAI-1), urokinase plasminogen activator (uPA), urokinase plasminogen activator receptor (uPAR), matrix metalloproteinase-3 (MMP-3) and missing-in-metastasis (MTSS) and (d) detecting the presence of one or more polypeptides or nucleic acids selected from the group consisting of plasminogen activator inhibitor 1 (PAI-1), urokinase plasminogen activator (uPA), urokinase plasminogen activator receptor (uPAR), matrix metalloproteinase-3 (MMP-3) and missing-in-metastasis (MTSS) when a complex is formed between the probe and the one or more polypeptides or nucleic acids.

38. A complex comprising (a) a probe and (b) a cmvIL10 protein or nucleic acid, wherein the cmvIL10 protein or nucleic acid is derived from a biological sample from an individual diagnosed with breast cancer, wherein the individual is infected with human cytomegalovirus (HCMV) but has not undergone seroconversion.

39. The complex of claim 39, wherein the probe comprises one or more nucleic acids.

40. The complex of claim 40, wherein the one or more nucleic acids specifically hybridize to a nucleic acid of SEQ ID NO: 1 or SEQ ID NO: 2.

41. The complex of any one of claims 40-41, wherein said one or more nucleic acids are PCR primers and PCR is performed subsequent to the complex forming between the PCR primers and the cmvIL-10 nucleic acid.

42. The complex of any one of claims 40-42, wherein the one or more nucleic acids is detectably labeled.

43. The complex of claim 39, wherein the probe comprises an antibody or fragment thereof.

44. The complex of claim 44, wherein the antibody or fragment thereof comprises a monoclonal antibody.

45. The complex of claim 44, wherein the antibody comprises a polyclonal antibody.

46. The complex of claim 46, wherein the polyclonal antibody is produced using a recombinantly-produced cmvIL-10 polypeptide immunogen comprising A26 to K176 of SEQ ID NO:3.

47. The complex of claim 46 or 47, wherein the polyclonal antibody is derived from goat.
